(19) United States
(12) Patent Application Publication
(10) Pub. No.: US 2010/0056431 A1
(43) Pub. Date: Mar. 4, 2010

(54) MEDICAL USES

(75) Inventors: Mona Stahle, Stockholm (SE); Gunther Weber, Solna (SE)

Correspondence Address: BIRCH STEWART KOLASCH & BIRCH, PO BOX 747, FALLS CHURCH, VA, 22040-0747, US

(73) Assignee: LIPOPEPTIDE AB, Stockholm (SE)

(21) Appl. No.: 12/305,601

(22) PCT Filed: Jun. 20, 2007
(86) PCT No.: PCT/GB2007/002289

§ 371 Date: Dec. 18, 2008

Related U.S. Application Data
(60) Provisional application No. 60/814,877, filed on Jun. 20, 2006.

(51) Int. Cl. A61K 38/12; A61K 38/16; A61K 38/02; A61K 38/08; A61K 38/10; A61P 35/00; A61K 38/07

(52) U.S. Cl. 514/9; 514/12; 514/2; 514/17; 514/16; 514/15; 514/14; 514/13; 514/18

(57) ABSTRACT

The present invention provides the use of a polypeptide comprising an amino acid sequence according to SEQ ID NO: 1 (corresponding to the N-terminal 25 amino acid fragment of the human antimicrobial protein, LL-37) or a biologically active fragment, variant, fusion or derivative thereof, in the preparation of a medicament for the treatment of cancer. In particular, the inventions provides the use such polypeptides, or a fragment, variant, fusion or derivative thereof, to inhibit the proliferation and/or metastasis of cancer cells, such as breast cancer cells. The invention further comprises methods for the treatment of cancer in a patient.

### FIELD OF INVENTION

The present invention relates to the use of a polypeptide comprising or consisting of the amino acid sequence of SEQ ID NO: 1, or a fragment, variant, fusion or derivative thereof, in the treatment of cancer. In particular, the invention provides methods for inhibiting the proliferation and/or metastasis of breast cancer cells.

### INTRODUCTION

Antimicrobial proteins are key effectors in the innate immune system. Human cathelicidin antimicrobial protein hCAP18, the only known cathelicidin in humans, consists of a conserved cathelin domain and a variable C-terminus, called LL-37 (Gudinundsson et al., 1996, Eur J Biochem 1238:325-32; Zanetti et al., 1995, FEBS Lett 374:1-5). Extracellular proteolytic processing of the holoprotein releases the LL-37 peptide, which has broad antimicrobial activity (Gudrnundsson et al., 1995, Proc Natl Acad Sci USA 92:7085-9; Agerberth et al., 1995, Proc Natl Acad Sci USA 92:195-99) as well as effects on host cells, some of which are mediated by the G-protein-coupled receptor, formyl peptide receptor-like 1 (FPRL1) (Yang et al., 2000, J Exp Med 192:1069-74; Koczulla et alt, 2003, J Clin Invest 111:1665-72). Human CAP18 is present in leucocytes (Cowland et al., 1995, FEBS Left 368:173-76) and is expressed in skin and other epithelia where it is upregulated in association with inflammation (Cowland et al., 1995, FEBS Lett 368:173-76; Frohm et al., 1997, J Biol Chem 272:15258-63) and injury (Dorschner et al., 2001, J Invest Dermatol 117:91-97; Heilborn et al., 2003, J Invest Dermatol 120:379-89) consistent with a role in innate barrier protection.

Some studies suggest that certain antimicrobial proteins may play a role in the non-specific host defence against tumours. For example, cecropin and melittin have been shown to exhibit antitumour activity in tumour derived cell lines (see Winder et al., 1998, Biochem Biophys Res Commun 242:608-12). Proline-rich antimicrobial peptide, PR-39, altered invasive activity and actin structure in human hepatocellular carcinoma cells (see Ohtake et al., 1999, Br J Cancer 181:393-403.).

In contrast, recent data suggest that hCAP18/LL-37 may promote rather than inhibit tumour cell growth in breast cancer (see Heilborn et at., 2005, Int. J. Cancer 114:713-9).

### SUMMARY OF INVENTION

A first aspect of the invention provides the use of a polypeptide comprising an amino acid sequence according to SEQ ID NO: 1 or a biologically active fragment, variant, fusion or derivative thereof, in the preparation of a medicament for the treatment of cancer.

| | |
|---|---|
| LLGDFFRKSKEKIGKEFKRIVQRIK | [SEQ ID NO: 1] |

In a preferred embodiment, the medicament is capable of inhibiting the proliferation of cancer cells.

By “proliferation” we include an increase in the number and/or size of cancer cells.

Alternatively, or preferably in addition, the medicament is capable of inhibiting metastasis of cancer cells.

By ‘metastasis’ we mean the movement or migration (e.g. invasiveness) of cancer cells from a primary tumour site in the body of a subject to one or more other areas within the subject's body (where the cells can then form secondary tumours). Thus, in one embodiment the invention provides agents and methods for inhibiting, in whole or in part, the formation of secondary tumours in a subject with cancer. It will be appreciated by skilled persons that the effect of an agent as described herein on ‘metastasis’ is distinct from any effect such agents may or may not have on cancer cell proliferation.

Advantageously, the medicament to be capable of inhibiting the proliferation and/or metastasis of cancer cells selectively.

By ‘selectively’ we mean that the medicament inhibits the proliferation and/or metastasis of cancer cells to a greater extent than it modulates the function (e.g. proliferation) of non-cancer cells. Preferably, the medicament inhibits the proliferation and/or metastasis of cancer cells only. Thus, we exclude medicaments which have a non-specific effect on cell function.

It will also be appreciated by persons skilled in the art that inhibition of the proliferation and/or metastasis of cancer cells may be in whole or in part. In a preferred embodiment, the medicament is capable of inhibiting the proliferation of cancer cells by 20% or more compared to the proliferation of cancer cells which have not been exposed to the medicament, for example by at least 30%, 40%, 50%, 60%, 70%, 80%, 90% or more. Alternatively, or preferably in addition, the medicament is capable of inhibiting metastasis of cancer cells by 20% or more compared to metastasis of cancer cells which have not been exposed to the medicament, for example by at least 30%, 40%, 50%, 60%, 70%, 80%, 90% or more.

The medicaments are suitable for use in the treatment of any cancer type. However, it is particularly preferred if the cancer cells are epithelial cells or squamous cells.

For example, the cancer cells may be selected from the group consisting of cancer cells of the breast, bile duct, brain, colon, stomach, reproductive organs, lung and airways, skin, gallbladder, liver, nasopharynx, nerve cells, kidney, prostate, lymph glands and gastrointestinal tract.

Preferably, the cancer cells are breast cancer cells. More preferably, the breast cancer cells are Elston grade III cells. Most preferably, the breast cancer cells are metastatic.

In a preferred embodiment of the first aspect of the invention, the polypeptide comprises or consists of an amino acid sequence according to SEQ ID NO: 1. This sequence, referred to as LL-25, corresponds to the N-terminal 25 amino acids of the C-terminal LL-37 peptide derived from the human cathelicidin antimicrobial protein hCAP18 (see Accession Nos. NP_{—}004336 and AAH55089).

The term ‘amino acid’ as used herein includes the standard twenty genetically-encoded amino acids and their corresponding stereoisomers in the ‘D’ form (as compared to the natural ‘L’ form), omega-amino acids and other naturally-occurring amino acids, unconventional amino acids (e.g. α,α-disubstituted amino acids, N-alkyl amino acids, etc.) and chemically derivatised amino acids (see below).

Preferably, however, the polypeptide, or fragment, variant, fusion or derivative thereof, comprises or consists of L-amino acids.

When an amino acid is being specifically enumerated, such as ‘alanine’ or ‘Ala’ or ‘A’, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

In a particularly preferred embodiment, the medicament comprises a polypeptide consisting of an amino acid sequence according to SEQ ID NO: 1.

In an alternative embodiment, the medicament comprises a biologically active fragment, variant, fusion or derivative of the amino acid sequence according to SEQ ID NO: 1.

By “biologically active” we mean that the fragment, variant fusion or derivative retains an anticancer activity of the amino acid sequence according to SEQ ID NO: 1. For example, the fragment, variant, fusion or derivative may retain the ability to inhibit, at least in part, the proliferation and/or metastasis of cancer cells. The retention of such biological activity may be determined using methods well known in the art (see Examples). For example, inhibition of the phosphorylation of MAPK may be used as a marker of anticancer activity of the fragment, variant, fusion or derivative. A further suitable method is the suppression of the metastatic phenotype of cancer cell colonies in semisolid agar, which can be induced by LL-37, or the suppression of LL-37-induced cell migration. In vivo, the suppression of metastasis in SCID mice treated with breast cancer cells overexpressing LL-37 can also be monitored.

It will be appreciated by persons skilled in the art that the polypeptide for use in the first aspect of the invention may comprise a biologically active fragment, variant, fusion or derivative of the LL-37 protein.

The amino acid sequence of LL-37 is shown below in SEQ ID NO:2:

[SEQ ID NO: 2]

LLGDFFRKSKEKIGKEFKRIVQRIKDFLRNLVPRTES

In a preferred embodiment, the polypeptide or biologically active fragment, variant, fusion or derivative thereof is provided for use at subcytotoxic doses. By “subcytotoxic” we mean the polypeptide or fragment, variant, fusion or derivative thereof is provided at a dose which does not have a direct cytotoxic effect on host cells, i.e. does not itself kill cells (preferably human cells). However, it will be appreciated that a low level of cytotoxicity may be present; hence, by subcytotoxic dose we include a dose which kills less than 5% of cells, preferably less than 4%, 3%, 2%, 1% of cells and most preferably a dose which kills none of the cells. Cytotoxicity may be determined using methods well known in the art (for example, an MTT assay; see Li et al., 2006, J. Am. Chem. Soc. 128:5776-5785; the relevant disclosures in which document are hereby incorporated by reference).

In an additional preferred embodiment, the invention does not encompass the use of the full-length LL-37 peptide nor does it encompass the use of N-terminal fragments of the LL-37 peptide greater than 25 amino acids in length.

In an alternative preferred embodiment, where the invention relates to the use of N-terminal fragments of the LL-37 peptide greater than 25 amino acids in length, the medicament is for the treatment of breast cancer.

Preferably, the fragment, variant, fusion or derivative does not comprise an amino acid sequence corresponding to amino acids 17 to 29 of human LL-37 peptide (see underlined amino acids in SEQ ID NO.2 below).

[SEQ ID NO: 2]

LLGDFFRKSKEKIGKEFKRIVQRIKDFLRNLVPRTES

Thus, in a preferred embodiment of the first aspect of the invention, the polypeptide comprises or consists of a fragment of the amino acid sequence according to SEQ ID NO: 1.

Advantageously, the fragment comprises or consists of at least 5 contiguous amino acids of SEQ ID NO: 1, for example at least 6, 7, 8, 9, 10, 11, 12, 13, 14, 15, 16, 17, 18, 19, 20, 21, 22, 23 or 24 contiguous amino acids of SEQ ID NO: 1. Thus, the fragment may comprise at least 6, 7, 8, 9, 10, 11, 12, 13, 14, 15, 16, 17, 18, 19, 20, 21, 22, 23 or 24 contiguous amino acids from the N-terminal (i.e. left) of SEQ ID NO: 1.

Conveniently, the fragment may comprise or consist of amino acids 2 to 25 of SEQ ID NO: 1, for example amino acids 3 to 25, 4 to 25, 5 to 25, 6 to 25, 7 to 25, 8 to 25, 9 to 25, 10 to 25, 11 to 25, 12 to 25, 13 to 25, 14 to 25, 15 to 25, 16 to 25, 17 to 25, 18 to 25, 19 to 25, 20 to 25 or 21 to 25 of SEQ ID NO: 1.

Alternatively, the fragment may comprise or consist of:
(a) Amino acid 1, 2, 3, 4, 5, 6, 7, 8, 9, 10, 11, 12, 13, 14, 15, 16, 17, 18, 19 or 20 to amino acid 24 of SEQ ID NO: 1;
(b) Amino acid 1, 2, 3, 4, 5, 6, 7, 8, 9, 10, 11, 12, 13, 14, 15, 16, 17, 18 or 19 to amino acid 23 of SEQ ID NO: 1;
(c) Amino acid 1, 2, 3, 4, 5, 6, 7, 8, 9, 10, 11, 12, 13, 14, 15, 16, 17 or 18 to amino acid 22 of SEQ ID NO: 1;
(d) Amino acid 1, 2, 3, 4, 5, 6, 7, 8, 9, 10, 11, 12, 13, 14, 15, 16 or 17 to amino acid 21 of SEQ ID NO: 1;
(e) Amino acid 1, 2, 3, 4, 5, 6, 7, 8, 9, 10, 11, 12, 13, 14, 15 or 16 to amino acid 20 of SEQ ID NO: 1;
(f) Amino acid 1, 2, 3, 4, 5, 6, 7, 8, 9, 10, 11, 12, 13, 14 or 15 to amino acid 19 of SEQ ID NO: 1;
(g) Amino acid 1, 2, 3, 4, 5, 6, 7, 8, 9, 10, 11, 12, 13 or 14 to amino acid 18 of SEQ ID NO: 1;
(h) Amino acid 1, 2, 3, 4, 5, 6, 7, 8, 9, 10, 11, 12 or 13 to amino acid 17 of SEQ ID NO: 1;
(i) Amino acid 1, 2, 3, 4, 5, 6, 7, 8, 9, 10, 11 or 12 to amino acid 16 of SEQ ID NO: 1;
(j) Amino acid 1, 2, 3, 4, 5, 6, 7, 8, 9, 10 or 11 to amino acid 15 of SEQ ID NO: 1;
(k) Amino acid 1, 2, 3, 4, 5, 6, 7, 8, 9 or 10 to amino acid 14 of SEQ ID NO: 1;
(l) Amino acid 1, 2, 3, 4, 5, 6, 7, 8 or 9 to amino acid 13 of SEQ ID NO: 1;
(m) Amino acid 1, 2, 3, 4, 5, 6, 7 or 7 to amino acid 12 of SEQ ID NO: 1;
(n) Amino acid 1, 2, 3, 4, 5, 6 or 7 to amino acid 11 of SEQ ID NO: 1;
(O) Amino acid 1, 2, 3, 4, 5 or 6 to amino acid 10 of SEQ ID NO: 1;
(p) Amino acid 1, 2, 3, 4 or 5 to amino acid 9 of SEQ ID NO: 1;
(q) Amino acid 1, 2, 3 or 4 to amino acid 8 of SEQ ID NO: 1;
(r) Amino acid 1, 2 or 3 to amino acid 7 of SEQ ID NO: 1;
(s) Amino acid 1 or 2 to amino acid 6 of SEQ ID NO: 1; or
(t) Amino acid 1 to amino acid 5 of SEQ ID NO: 1.

Most preferably, the fragment comprises or consists of amino acids 17 to 25 of SEQ ID NO: 1, for example amino acids 17 to 24, 17 to 23, 17 to 22, 17 to 21 or 17 to 20 of SEQ ID NO: 1.

In an alternative embodiment of the first aspect of the invention, the polypeptide comprises or consists of a variant of the amino acid sequence according to SEQ ID NO: 1.

By ‘variant’ of the polypeptide we include insertions, deletions and substitutions, either conservative or non-conservative. For example, the variant polypeptide may be a non-naturally occurring variant.

It is particularly preferred that the variant has an amino acid sequence which has at least 50% identity with the amino acid sequence according to SEQ ID NO: 1 or a fragment thereof, for example at least 55%, 60%, 65%, 70%, 75%, 80%, 90%, 95%, 96%, 97%, 98% or at least 99% identity.

The percent sequence identity between two polypeptides may be determined using suitable computer programs, for example the GAP program of the University of Wisconsin Genetic Computing Group, and it will be appreciated that percent identity is calculated in relation to polypeptides whose sequences have been aligned optimally.

The alignment may alternatively be carried out using the Clustal W program (as described in Thompson et al., 1994, Nuc. Acid Res. 22:4673-4680, the relevant disclosures in which document are hereby incorporated by reference).

The parameters used may be as follows:
Fast pairwise alignment parameters: K-tuple(word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alternatively, the BESTFIT program may be used to determine local sequence alignments.

Variants may be made using the methods of protein engineering and site-directed mutagenesis well known in the art (see example, see Molecular Cloning: a Laboratory Manual, 3rd edition, Sambrook & Russell, 2001, Cold Spring Harbor Laboratory Press, the relevant disclosures in which document are hereby incorporated by reference).

In a further alternative embodiment of the first aspect of the invention, the medicament comprises or consists of a fusion protein.

By ‘fusion’ of a protein or polypeptide we include a polypeptide fused to any other polypeptide. For example, the said polypeptide may be fused to a polypeptide such as glutathione-S-transferase (GST) or protein A in order to facilitate purification of said polypeptide. Examples of such fusions are well known to those skilled in the art. Similarly, the said polypeptide may be fused to an oligo-histidine tag such as His6 or to an epitope recognised by an antibody such as the well-known Myc tag epitope. Fusions to any fragment, variant or derivative of said polypeptide are also included in the scope of the invention. It will be appreciated that fusions (or variants or derivatives thereof) which retain desirable properties, namely anticancer activity are preferred. It is also particularly preferred if the fusions are ones which are suitable for use in the methods described herein.

For example, the fusion may comprise a further portion which confers a desirable feature on the said polypeptide of the invention; for example, the portion may be useful in detecting or isolating the polypeptide, or promoting cellular uptake of the polypeptide. The portion may be, for example, a biotin moiety, a radioactive moiety, a fluorescent moiety, for example a small fluorophore or a green fluorescent protein (GFP) fluorophore, as well known to those skilled in the art. The moiety may be an immunogenic tag, for example a Myc tag, as known to those skilled in the art or may be a lipophilic molecule or polypeptide domain that is capable of promoting cellular uptake of the polypeptide, as known to those skilled in the art.

It will be appreciated by skilled persons that the polypeptide, or fragment, variant, fusion or derivative thereof, may comprise one or more amino acids that are modified or derivatised.

Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be further appreciated by persons skilled in the art that peptidomimetic compounds may also be useful. Thus, by ‘polypeptide’ we include peptidomimetic compounds which exhibit anticancer activity. The term ‘peptidomimetic’ refers to a compound that mimics the conformation and desirable features of a particular polypeptide as a therapeutic agent.

For example, the polypeptides described herein include not only molecules in which amino acid residues are joined by peptide (—CO—NH—) linkages but also molecules in which the peptide bond is reversed. Such retro-inverso peptidomimetics may be made using methods known in the art, for example such as those described in Meziere et al. (1997) J. Immunol. 159, 3230-3237, the relevant disclosures in which document are hereby incorporated by reference. This approach involves making pseudopeptides containing changes involving the backbone, and not the orientation of side chains. Retro-inverse peptides, which contain NH—CO bonds instead of CO—NH peptide bonds, are much more resistant to proteolysis. Alternatively, the polypeptide of the invention may be a peptidomimetic compound wherein one or more of the amino acid residues are linked by a -y(CH₂NH)— bond in place of the conventional amide linkage.

In a further alternative, the peptide bond may be dispensed with altogether provided that an appropriate linker moiety which retains the spacing between the carbon atoms of the amino acid residues is used; it is particularly preferred if the linker moiety has substantially the same charge distribution and substantially the same planarity as a peptide bond.

It will be appreciated that the polypeptide may conveniently be blocked at its N- or C-terminus so as to help reduce susceptibility to exoproteolytic digestion, e.g. by amidation.

A variety of uncoded or modified amino acids such as D-amino acids and N-methyl amino acids have also been used to modify mammalian peptides. In addition, a presumed bioactive conformation may be stabilised by a covalent modification, such as cyclisation or by incorporation of lactam or other types of bridges, for example see Veber et al., 1978, Proc. Natl. Acad. Sci. USA 75:2636 and Thursell et al., 1983, Biochem. Biophys. Res. Comm. 111:166, the relevant disclosures in which documents are hereby incorporated by reference.

A common theme among many of the synthetic strategies has been the introduction of some cyclic moiety into a peptide-based framework. The cyclic moiety restricts the conformational space of the peptide structure and this frequently results in an increased affinity of the peptide for a particular biological receptor. An added advantage of this strategy is that the introduction of a cyclic moiety into a peptide may also result in the peptide having a diminished sensitivity to cellular peptidases.

Thus, preferred polypeptides comprise terminal cysteine amino acids. Such a polypeptide may exist in a heterodetic cyclic form by disulphide bond formation of the mercaptide groups in the terminal cysteine amino acids or in a homodetic form by amide peptide bond formation between the terminal amino acids. As indicated above, cyclising small peptides through disulphide or amide bonds between the N- and C-terminus cysteines may circumvent problems of affinity and half-life sometime observed with linear peptides, by decreasing proteolysis and also increasing the rigidity of the structure, which may yield higher affinity compounds. Polypeptides cyclised by disulphide bonds have free amino and carboxy-termini which still may be susceptible to proteolytic degradation, while peptides cyclised by formation of an amide bond between the N-terminal amine and C-terminal carboxyl and hence no longer contain free amino or carboxy termini. Thus, the peptides of the present invention can be linked either by a C—N linkage or a disulphide linkage.

The present invention is not limited in any way by the method of cyclisation of peptides, but encompasses peptides whose cyclic structure may be achieved by any suitable method of synthesis. Thus, heterodetic linkages may include, but are not limited to formation via disulphide, alkylene or sulphide bridges. Methods of synthesis of cyclic homodetic peptides and cyclic heterodetic peptides, including disulphide, sulphide and alkylene bridges, are disclosed in U.S. Pat. No. 5,643,872. Other examples of cyclisation methods are discussed and disclosed in U.S. Pat. No. 6,008,058, the relevant disclosures in which documents are hereby incorporated by reference.

A further approach to the synthesis of cyclic stabilised peptidomimetic compounds is ring-closing metathesis (RCM). This method involves steps of synthesising a peptide precursor and contacting it with an RCM catalyst to yield a conformationally restricted peptide. Suitable peptide precursors may contain two or more unsaturated C—C bonds. The method may be carried out using solid-phase-peptide-synthesis techniques. In this embodiment, the precursor, which is anchored to a solid support, is contacted with a RCM catalyst and the product is then cleaved from the solid support to yield a conformationally restricted peptide.

Another approach, disclosed by D. H. Rich in Protease Inhibitors, Barrett and Selveson, eds., Elsevier (1986; the relevant disclosures in which document are hereby incorporated by reference), has been to design peptide mimics through the application of the transition state analogue concept in enzyme inhibitor design. For example, it is known that the secondary alcohol of staline mimics the tetrahedral transition state of the scissile amide bond of the pepsin substrate.

In summary, terminal modifications are useful, as is well known, to reduce susceptibility by proteinase digestion and therefore to prolong the half-life of the peptides in solutions, particularly in biological fluids where proteases may be present. Polypeptide cyclisation is also a useful modification and is preferred because of the stable structures formed by cyclisation and in view of the biological activities observed for cyclic peptides.

Thus, in one embodiment the polypeptide, or fragment, variant, fusion or derivative thereof, is cyclic.

However, in an alternative embodiment, the polypeptide, or fragment, variant, fusion or derivative thereof, is linear.

The present invention also includes the use of medicaments comprising pharmaceutically acceptable acid or base addition salts of the above described polypeptides. The acids which are used to prepare the pharmaceutically acceptable acid addition salts of the aforementioned base compounds useful in this invention are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others.

Pharmaceutically acceptable base addition salts may also be used to produce pharmaceutically acceptable salt forms of the polypeptides. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others.

The polypeptide, or fragment, variant, fusion or derivative thereof, may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of activity loss and that use levels may have to be adjusted upward to compensate. Preferably, the lyophilised (freeze dried) polypeptide loses no more than about 20%, or no more than about 25%, or no more than about 30%, or no more than about 35%, or no more than about 40%, or no more than about 45%, or no more than about 50% of its activity (prior to lyophilisation) when rehydrated.

In a further aspect of the invention there is provided a combination product comprising:
(A) a first agent which comprises of consists of a polypeptide or fragment, variant, fusion or derivative thereof, as defined above in relation to the first aspect of the invention; and
(B) a second agent that inhibits the biological activity of an epidermal growth factor (EGF) receptor,
   wherein each of components (A) and (B) is formulated in admixture with a pharmaceutically acceptable adjuvant, diluent or carrier.

By an ‘agent’ we include all chemical entities, for example oligonucleotides, polynucleotide, polypeptides, peptidomimetics and small compounds.

In a preferred embodiment the combination product of the invention comprises a pharmaceutical formulation including a first agent as defined above, a second agent that inhibits the biological activity of an EGF receptor, and a pharmaceutically-acceptable adjuvant, diluent or carrier.

In an alternative embodiment the combination product of the invention comprises a kit of parts comprising components:
(A) a pharmaceutical formulation including a first agent which comprises of consists of a polypeptide or fragment, variant, fusion or derivative thereof, as defined above, in admixture with a pharmaceutically-acceptable adjuvant, diluent or carrier; and
(B) a pharmaceutical formulation including a second agent that inhibits the activity of EGF receptors, in admixture with a pharmaceutically-acceptable adjuvant, diluent or carrier,
   which components (A) and (B) are each provided in a form that is suitable for administration in conjunction with the other.

By bringing the two components “into association with” each other, we include that components (A) and (B) of the kit of parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Thus, in respect of the combination product according to the invention, the term “administration in conjunction with” includes that the two components of the combination product (the first agent and the second agent) are administered (optionally repeatedly), either together, or sufficiently closely in time, to enable a beneficial effect for the patient, that is greater, over the course of the treatment of the relevant condition, than if either a formulation comprising the first agent as defined above, or a formulation comprising the second agent that inhibits the activity of EGF receptors, are administered (optionally repeatedly) alone, in the absence of the other component, over the same course of treatment. Determination of whether a combination provides a greater beneficial effect in respect of, and over the course of treatment of, a particular condition will depend upon the condition to be treated or prevented, but may be achieved routinely by the skilled person.

Preferably the EGF receptor being inhibited is selected from the group consisting of FrbB1 (EGF-R), ErbB2, ErbB3 and ErbB4. Most preferably, the EGF receptor is ErbB2.

Advantageously, the second agent inhibits the biological activity of an EGF receptor by altering the transcription, translation and/or binding properties of an EGF receptor.

Such agents may be identified using methods well known in the art, such as:
(a) by determining the effect of a test agent on levels of expression of EGF receptor mRNA, for example by Northern blotting or quantitative RT-PCR;
(b) by determining the effect of a test agent on levels of EGF receptor protein, for example by immunoassays using anti-EGF receptor antibodies; and
(c) by determining the effect of a test agent on a functional marker of EGF receptor activity, for example phosphorylation of ErbB2.

In a preferred embodiment, the second agent is an inhibitor of the transcription of an EGF receptor.

In an alternative embodiment, the second agent is an inhibitor of the translation of an EGF receptor.

In a further alternative embodiment, the second agent is an inhibitor of the binding properties of an EGF receptor.

In a further alternative embodiment, the second agent is an EGF receptor antagonist. It will be appreciated by persons skilled in the art that the agent(s) may inhibit biological activity of by blocking receptor function directly, i.e. by acting as a receptor antagonist, or indirectly.

It will be appreciated by persons skilled in the art that inhibition of the EGF receptors by the second agent may be in whole or in part. For example, the agent may inhibit the biological activity of EGF receptors by at least 10%, preferably at least 20%, 30%, 40%, 50%, 60%, 70%, 80% or 90%, and most preferably by 100% compared to the biological activity of EGF receptors and/or EGF receptors in cancer cells which have not been exposed to the agent

Advantageously, the second agent is capable of inhibiting the biological activity of ERBB2 by 50% or more compared to the biological activity of ERBB2 in cancer cells which have not been exposed to the agent.

Preferably, the second agent is selected from the group consisting of short interfering RNA (siRNA) molecules, antisense oligonucleotides, compounds with binding affinity for EGF receptors and small inhibitor compounds.

Particularly preferred examples of EGF receptor inhibitors include the drug Herceptin (trastuzumab, Genentech) a monoclonal antibody with specificity for ErbB2, the drug Erbitux (cetuximab, Bristol-Meyers Squibb) a monoclonal antibody with specificity for EGF-R (ErbB1)), other monoclonal antibodies such as MAB225, the small molecule IRESSA (gefitinib, Astra Zeneca) that inhibits EGF receptors by inhibiting tyrosine kinases and other tyrosine kinase inhibitors (e.g. PD153035, GW572016 and others, available commercially from suppliers such as Calbiochem/Merck).

Methods for the production of polypeptides, or fragment, variant, fusion or derivative thereof, for use in the first aspect of the invention are well known in the art. Conveniently, the polypeptide, or fragment, variant, fusion or derivative thereof, is or comprises a recombinant polypeptide.

Thus, a nucleic acid molecule (or polynucleotide) encoding the polypeptide, or fragment, variant, fusion or derivative thereof, may be expressed in a suitable host and the polypeptide obtained therefrom. Suitable methods for the production of such recombinant polypeptides are well known in the art (for example, see Sambrook & Russell, 2000, Molecular Cloning, A Laboratory Manual, Third Edition, Cold Spring Harbor, N.Y., the relevant disclosures in which document are hereby incorporated by reference).

In brief, expression vectors may be constructed comprising a nucleic acid molecule which is capable, in an appropriate host, of expressing the polypeptide encoded by the nucleic acid molecule.

A variety of methods have been developed to operably link nucleic acid molecules, especially DNA, to vectors, for example, via complementary cohesive termini. For instance, complementary homopolymer tracts can be added to the DNA segment to be inserted into the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, e.g. generated by endonuclease restriction digestion, is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerising activities.

The combination of these activities therefore generates blunt-ended DNA segments. The blunt-ended segments are then incubated with a larger molar excess of linker molecules in the presence of an enzyme that is able to catalyse the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease site are commercially available from a number of sources including International Biotechnologies Inc., New Haven, Conn., USA.

A desirable way to modify the DNA encoding the polypeptide of the invention is to use PCR. This method may be used for introducing the DNA into a suitable vector, for example by engineering in suitable restriction sites, or it may be used to modify the DNA in other useful ways as is known in the art.

In this method the DNA to be enzymatically amplified is flanked by two specific primers which themselves become incorporated into the amplified DNA. The said specific primers may contain restriction endonuclease recognition sites which can be used for cloning into expression vectors using methods known in the art.

The DNA (or in the case of retroviral vectors, RNA) is then expressed in a suitable host to produce a polypeptide. Thus, the DNA encoding the polypeptide may be used in accordance with known techniques, appropriately modified in view of the teachings contained herein, to construct an expression vector, which is then used to transform an appropriate host cell for the expression and production of the compound of the invention or binding moiety thereof. Such techniques include those disclosed in U.S. Pat. Nos. 4,440,859 issued 3 Apr. 1984 to Rutter et al, 4,530,901 issued 23 Jul. 1985 to Weissman, 4,582,800 issued 15 Apr. 1986 to Crowl, 4,677,063 issued 30 Jun. 1987 to Mark et al, 4,678,751 issued 7 Jul. 1987 to Goeddel, 4,704,362 issued 3 Nov. 1987 to Itakura et al, 4,710,463 issued 1 Dec. 1987 to Murray, 4,757,006 issued 12 Jul. 1988 to Toole, Jr. et al, 4,766,075 issued 23 Aug. 1988 to Goeddel et al and 4,810,648 issued 7 Mar. 1989 to Stalker, all of which are incorporated herein by reference.

The DNA (or in the case or retroviral vectors, RNA) encoding the polypeptide may be joined to a wide variety of other DNA sequences for introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. The vector is then introduced into the host through standard techniques. Generally, not all of the hosts will be transformed by the vector. Therefore, it will be necessary to select for transformed host cells. One selection technique involves incorporating into the expression vector a DNA sequence, with any necessary control elements, that codes for a selectable trait in the transformed cell, such as antibiotic resistance. Alternatively, the gene for such selectable trait can be on another vector, which is used to co-transform the desired host cell.

Host cells that have been transformed by the expression vector are then cultured for a sufficient time and under appropriate conditions known to those skilled in the art in view of the teachings disclosed herein to permit the expression of the polypeptide, which can then be recovered.

Many expression systems are known, including bacteria (for example, E. coli and Bacillus subtilis), yeasts (for example Saccharomyces cerevisiae), filamentous fungi (for example Aspergillus), plant cells, animal cells and insect cells.

The vectors typically include a prokaryotic replicon, such as the ColE1 ori, for propagation in a prokaryote, even if the vector is to be used for expression in other, non-prokaryotic, cell types. The vectors can also include an appropriate promoter such as a prokaryotic promoter capable of directing the expression (transcription and translation) of the genes in a bacterial host cell, such as E. coli, transformed therewith.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a DNA segment.

Typical prokaryotic vector plasmids are pUC18, pUC19, pBR322 and pBR329 available from Biorad Laboratories, (Richmond, Calif., USA) and pTrc99A and pKK223-3 available from Pharmacia, Piscataway, N.J., USA.

A typical mammalian cell vector plasmid is pSVL available from Pharmacia, Piscataway, N.J., USA. This vector uses the SV40 late promoter to drive expression of cloned genes, the highest level of expression being found in T antigen-producing cells, such as COS-1 cells.

An example of an inducible mammalian expression vector is pMSG, also available from Pharmacia. This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, TRP1, LEU2 and URA3. Plasmid pRS413-416 is a Yeast Centromere plasmids (Ycps).

Other vectors and expression systems are well known in the art for use with a variety of host cells.

The host cell can be either prokaryotic or eukaryotic. Bacterial cells are preferred prokaryotic host cells and typically are a strain of E. coli such as, for example, the E. coli strains DH5 available from Bethesda Research Laboratories Inc., Bethesda, Md., USA, and RR1 available from the American Type Culture Collection (ATCC) of Rockville, Md., USA (No. ATCC 31343). Preferred eukaryotic host cells include yeast, insect and mammalian cells, preferably vertebrate cells such as those from a mouse, rat, monkey or human fibroblastic and kidney cell lines. Yeast host cells include YPH499, YPH500 and —YTH501 which are generally available from Stratagene Cloning Systems, La Jolla, Calif. 92037, USA. Preferred mammalian host cells include Chinese hamster ovary (CHO) cells available from the ATCC as CRL 1658 and 293 cells which are human embryonic kidney cells. Preferred insect cells are Sf9 cells which can be transfected with baculovirus expression vectors.

Transformation of appropriate cell hosts with a DNA construct is accomplished by well known methods that typically depend on the type of vector used. With regard to transformation of prokaryotic host cells, see, for example, Cohen et al. (1972) Proc. Natl. Acad. Sci. USA 69, 2110 and Molecular Cloning: a Laboratory Manual, 3rd edition, Sambrook & Russell, 2001, Cold Spring Harbor Laboratory Press. Transformation of yeast cells is described in Sherman et al (1986) Methods In Yeast Genetics, A Laboratory Manual, Cold Spring Harbor, N.Y. The method of Beggs (1978) Nature 275, 104-109 is also useful. With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA. The relevant disclosures in the above documents are hereby incorporated by reference.

Electroporation is also useful for transforming and/or transfecting cells and is well known in the art for transforming yeast cells, bacterial cells, insect cells and vertebrate cells.

For example, many bacterial species may be transformed by the methods described in Luchansky et al (1988) Mol. Microbiol. 2, 637-646, the relevant disclosures in which document are hereby incorporated by reference. The greatest number of transformants is consistently recovered following electroporation of the DNA-cell mixture suspended in 2.5 PEB using 6250V per cm at 25 μFD.

Methods for transformation of yeast by electroporation are disclosed in Becker & Guarente (1990) Methods Enzymol, 194, 182, the relevant disclosures in which document are hereby incorporated by reference.

Successfully transformed cells, i.e. cells that contain a DNA construct encoding a polypeptide, can be identified by well known techniques. For example, cells resulting from the introduction of an expression construct of the present invention can be grown to produce the polypeptide of the invention. Cells can be harvested and lysed and their DNA content examined for the presence of the DNA using a method such as that described by Southern (1975) J. Mol. Biol. 98, 503 or Berent et al (1985) Biotech. 3, 208, the relevant disclosures in which document are hereby incorporated by reference. Alternatively, the presence of the protein in the supernatant can be detected using antibodies.

In addition to assaying directly for the presence of recombinant DNA, successful transformation can be confirmed by well known immunological methods when the recombinant DNA is capable of directing the expression of the protein. For example, cells successfully transformed with an expression vector produce proteins displaying appropriate antigenicity.

Samples of cells suspected of being transformed are harvested and assayed for the protein using suitable antibodies.

The host cell may be a host cell within a non-human animal body. Thus, transgenic non-human animals which express a polypeptide by virtue of the presence of the transgene are included. Preferably, the transgenic non-human animal is a rodent such as a mouse. Transgenic non-human animals can be made using methods well known in the art (see below).

Methods of cultivating host cells and isolating recombinant proteins are well known in the art. It will be appreciated that, depending on the host cell, the compounds of the invention (or binding moieties thereof) produced may differ. For example, certain host cells, such as yeast or bacterial cells, either do not have, or have different, post-translational modification systems which may result in the production of forms of compounds of the invention (or binding moieties thereof) which may be post-translationally modified in a different way.

It is preferred that polypeptides for use in the methods of the invention are produced in a eukaryotic system, such as a mammalian cell.

Polypeptides can also be produced in vitro using a commercially available in vitro translation system, such as rabbit reticulocyte lysate or wheatgerm lysate (available from Promega). Preferably, the translation system is rabbit reticulocyte lysate. Conveniently, the translation system may be coupled to a transcription system, such as the TNT transcription-translation system (Promega). This system has the advantage of producing suitable mRNA transcript from an encoding DNA polynucleotide in the same reaction as the translation.

Also described herein is a pharmaceutical composition comprising a polypeptide, or fragment, variant, fusion or derivative thereof, and a pharmaceutically acceptable excipient, diluent or carrier.

As used herein, ‘pharmaceutical composition’ means a therapeutically effective formulation for use in the methods of the invention.

A ‘therapeutically effective amount’, or ‘effective amount’, or ‘therapeutically effective’, as used herein, refers to that amount which provides a therapeutic effect for a given condition (cancer) and administration regimen. This is a predetermined quantity of active material calculated to produce a desired therapeutic effect in association with the required additive and diluent, i.e. a carrier or administration vehicle. Further, it is intended to mean an amount sufficient to reduce and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art.

The polypeptides can be formulated at various concentrations, depending on the efficacy/toxicity of the compound being used. Preferably, the formulation comprises the active agent at a concentration of between 0.1 μM and 1 mM, more preferably between 1 μM and 100 μM, between 5 μM and 50 μM, between 10 μM and 50 μM, between 20 μM and 40 μM and most preferably about 30 μM. For in vitro applications, formulations may comprise a lower concentration of a polypeptide, for example between 0.0025 μM and 1 μM.

Thus, the pharmaceutical formulation may comprise an amount of a polypeptide, or fragment, variant, fusion or derivative thereof sufficient to treat cancer.

It will be appreciated by persons skilled in the art that the medicaments generally be administered in admixture with a suitable pharmaceutical excipient diluent or carrier selected with regard to the intended route of administration and standard pharmaceutical practice (for example, see Remington: The Science and Practice of Pharmacy, 19^{th} edition, 1995, Ed. Alfonso Gennaro, Mack Publishing Company, Pennsylvania, USA, the relevant disclosures in which document are hereby incorporated by reference).

For example, the medicaments and agents can be administered orally, buccally or sublingually in the form of tablets, capsules, ovules, elixirs, solutions or suspensions, which may contain flavouring or colouring agents, for immediate- , delayed- or controlled-release applications. The medicaments and agents may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxyl-propylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the polypeptides may be combined with various sweetening or flavouring agents, colouring matter or dyes, with emulsifying and/or suspending agents and with diluents such as water, ethanol, propylene glycol and glycerin, and combinations thereof.

The medicaments can also be administered parenterally, for example, intravenously, intra-articularly, intra-arterially, intraperitoneally, intra-thecally, intraventricularly, intrasternally, intracranially, intramuscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example scaled ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the medicaments will usually be from 1 to 1000 mg per adult (i.e. from about 0.015 to 15 mg/kg), administered in single or divided doses. For example, a dose of 1 to 10 mg/kg may be used, such as 3 mg/kg.

The medicaments can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoro-methane, dichlorotetrafluoro-ethane, a hydrofluroalkane such as 1,1,1,2-tetrafluoroethane (HFA 134A3 or 1,1,1,2,3,3,3-heptafluoropropane (HFA 1227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or ‘puff’ contains at least 1 mg of a compound of the invention for delivery to the patient. It will be appreciated that the overall daily dose with an aerosol will vary from patient to patient, and may be administered in a single dose or, more usually, in divided doses throughout the day.

Alternatively, the medicaments can be administered in the form of a suppository or pessary, or they may be applied topically in the form of a lotion, solution, cream, ointment or dusting powder. The compounds of the invention may also be transdermally administered, for example, by the use of a skin patch. They may also be administered by the ocular route.

For application topically to the skin, the medicaments can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Where the medicament or agent is a polypeptide, it may be preferable to use a sustained-release drug delivery system, such as a microsphere. These are designed specifically to reduce the frequency of injections. An example of such a system is Nutropin Depot which encapsulates recombinant human growth hormone (rhGH) in biodegradable microspheres that, once injected, release rhGH slowly over a sustained period.

Sustained-release polypeptide compositions also include liposomally entrapped polypeptides. Liposomes containing the polypeptides are prepared by methods known per se. See, for example Epstein et al., Proc. Natl. Acad. Sci. USA 82: 3688-92 (1985); Hwang et al., Proc. Natl. Acad. Sci. USA 77: 4030-4 (1980); U.S. Pat. Nos. 4,485,045; 4,544,545; 6,139,869; and 6,027,726, the relevant disclosures in which documents are hereby incorporated by reference. Ordinarily, the liposomes are of the small (about 200 to about 800 Angstroms), unilamellar type in which the lipid content is greater than about 30 mole percent (mol. %) cholesterol; the selected proportion being adjusted for the optimal polypeptide therapy.

Alternatively, polypeptide medicaments and agents can be administered by a surgically implanted device that releases the drug directly to the required site.

Electroporation therapy (EPT) systems can also be employed for the administration of proteins and polypeptides. A device which delivers a pulsed electric field to cells increases the permeability of the cell membranes to the drug, resulting in a significant enhancement of intracellular drug delivery.

Proteins and polypeptides can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of protein and polypeptide delivery is the thermo-sensitive ReGel injectable. Below body temperature, ReGel is an injectable liquid while at body temperature it immediately forms a gel reservoir that slowly erodes and dissolves into known, safe, biodegradable polymers. The active drug is delivered over time as the biopolymers dissolve.

Protein and polypeptide pharmaceuticals can also be delivered orally. One such system employs a natural process for oral uptake of vitamin B12 in the body to co-deliver proteins and polypeptides. By riding the vitamin B12 uptake system, the protein or polypeptide can move through the intestinal wall. Complexes are produced between vitamin B12 analogues and the drug that retain both significant affinity for intrinsic factor (IF) in the vitamin B12 portion of the complex and significant bioactivity of the drug portion of the complex.

A second aspect of the invention provides a method for treating cancer in a patient, the method comprising administering to the patient a polypeptide comprising an amino acid sequence according to SEQ ID NO: 1, or a biologically active fragment, variant, fusion or derivative thereof, as defined above in relation to the first aspect of the invention.

Persons skilled in the art will further appreciate that the uses and methods of the present invention have utility in both the medical and veterinary fields. Thus, the medicaments may be used in the treatment of both human and non-human animals (such as horses, dogs and cats). Preferably, however, the patient is human.

By ‘treatment’ we include both therapeutic and prophylactic treatment of the patient. The term ‘prophylactic’ is used to encompass the use of a polypeptide or formulation described herein which either prevents or reduces the likelihood of cancer in a patient or subject.

As discussed above, the term ‘effective amount’ is used herein to describe concentrations or amounts of compounds according to the present invention which may be used to produce a favourable change in a disease or condition treated, whether that change is a remission, a favourable physiological result, a reversal or attenuation of a disease state or condition treated, the prevention or the reduction in the likelihood of a condition or disease state occurring, depending upon the disease or condition treated.

It will be appreciated that the medicaments described herein may be administered to patients in combination with one or more additional therapeutic agents, for example one or more conventional cancer treatments.

In a preferred embodiment, the polypeptide or fragment, variant, fusion or derivative thereof is capable of inhibiting the proliferation of cancer cells.

Alternatively, or preferably in addition, the polypeptide or fragment, variant, fusion or derivative thereof is capable of inhibiting metastasis of cancer cells.

Advantageously, the peptide or fragment, variant, fusion or derivative thereof to be capable of inhibiting the proliferation and/or metastasis of cancer cells selectively.

By ‘selectively’ we mean that the polypeptide or fragment, variant, fusion or to derivative thereof inhibits the proliferation and/or metastasis of cancer cells to a greater extent than it modulates the function (e.g. proliferation) of non-cancer cells. Preferably, the polypeptide or fragment, variant, fusion or derivative thereof inhibits the proliferation and/or metastasis of cancer cells only.

It will also be appreciated by persons skilled in the art that inhibition of the proliferation and/or metastasis of cancer cells may be in whole or in part. In a preferred embodiment, the polypeptide or fragment, variant, fusion or derivative thereof is capable of inhibiting the proliferation of cancer cells by 20% or more compared to the proliferation of cancer cells which have not been exposed to the medicament, for example by at least 30%, 40%, 50%, 60%, 70%, 80%, 90% or more. Alternatively, or preferably in addition, the polypeptide or fragment, variant, fusion or derivative thereof is capable of inhibiting metastasis of cancer cells by 20% or more compared to metastasis of cancer cells which have not been exposed to the medicament, for example by at least 30%, 40%, 50%, 60%, 70%, 80%, 90% or more.

As discussed above, the polypeptide or fragment, variant, fusion or derivative thereof is suitable for use in the treatment of any cancer type. However, it is particularly preferred if the cancer cells are epithelial cells or squamous cells.

For example, the cancer cells may be selected from the group consisting of cancer cells of the breast, bile duct brain, colon, stomach, reproductive organs, lung and airways, skin, gallbladder, liver, nasopharynx, nerve cells, kidney, prostate, lymph glands and gastrointestinal tract.

Preferably, the cancer cells are breast cancer cells. More preferably, the breast cancer cells are Elston grade III cells. Most preferably, the breast cancer cells are metastatic.

Preferred aspects of the invention are described in the following non-limiting examples, with reference to the following figures:

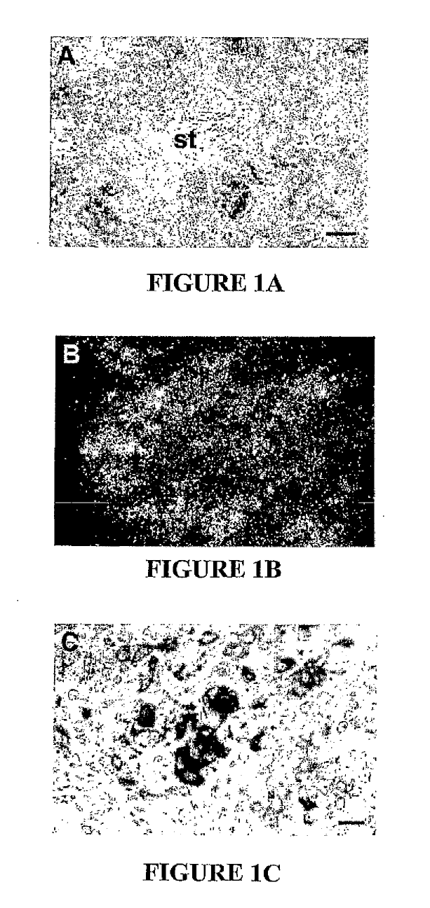

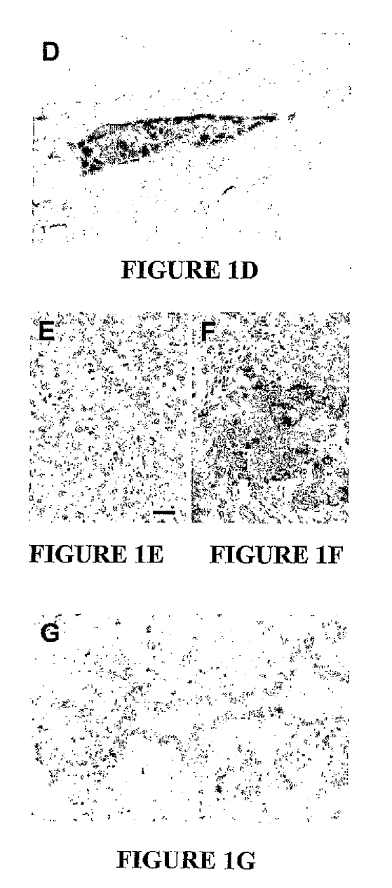

FIG. 1—hCAP18/LL-37 is highly expressed in breast cancer.

(a) Section of ductal breast carcinoma grade III (patient no 7, Table 2) demonstrating strong immunoreactivity for hCAP18 protein in tumour cells (red precipitate) surrounding a stromal island (st). (b) In situ hybridisation shows a matching signal for hCAP18 mRNA in a section from the same tissue. Intense autoradiographic signals appear as white grains under dark-field illumination. (c) High-power view of carcinoma cells demonstrates strongly immunoreactive cells adjacent to tumour cells devoid of immunoreactivity. (d) hCAP18 immunoreactive breast carcinoma cells within a blood vessel. (e) Immunoabsorption with cathelin recombinant peptide completely abolished the hCAP18 immunoreactivity (same tissue as FIG. 1a). (f) Regular immunostaining for hCAP18 as positive control during immunoabsorption (same tissue as FIG. 1a). (g) Normal mammary gland epithelium shows weak immunoreactivity for hCAP18. Photomicrographs (a, c-g) show results obtained with the hCAP18 antibody at 1:500 dilution. Scale bars (a, b)=100 μam; (c, d)=25 μm; (e, f, g)=10 μm.

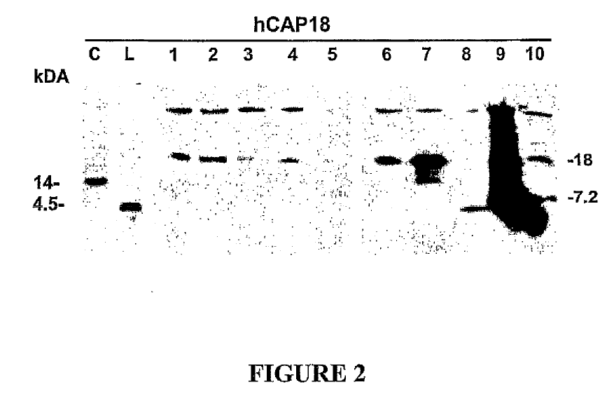

FIG. 2—hCAP-18/LL-37 is detected by immunoblotting in breast cancer.

Clinical data of patients are presented in Table 2 (sample 1-10). Recombinant cathelin (C) and LL-37 peptide (L) were used as size references. Normal breast tissue is presented in lane 1. Elston grade I tumours are presented in lanes 2, 4 and 5. A grade II tumour is presented in lane 3 and grade III tumours are presented in lanes 6-10. In all tissues there were immunoreactive bands corresponding to the intact non-processed 18 kDa holoprotein. The processed LL-37 peptide (4 kD) was visible in 4 of the 5 grade III tumours (no 7-10).

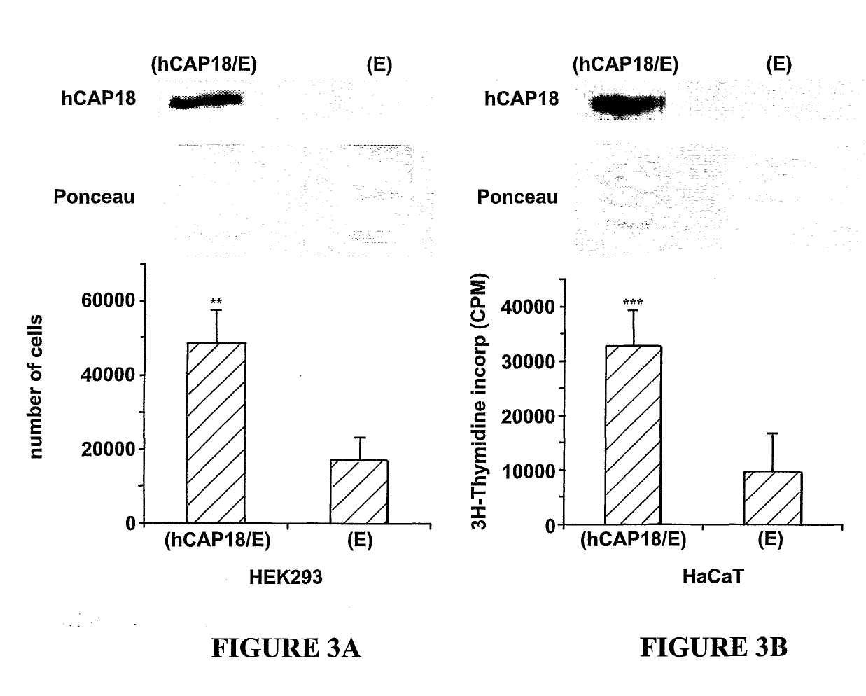

FIG. 3—Transgenic expression of hCAP18 in epithelial cells increases cell proliferation.

(a) Upper panel, left lane; Immunoblotting on HEK293 extracts with anti-LL37 antiserum. Cells transfected with a bicistronic vector hCAP18+EGFP (hCAP18/E) show hCAP18 protein expression. Upper panel, right lane; HEK293 cells transfected with only EGFP (E). Lower panel; HEK293 cells (hCAP18/E) demonstrate significantly higher proliferation rate (evaluated with is Flow-Cytometry) compared with control cells (E). Ponceau staining is shown as loading control. (b) Upper panel, left lane; HaCaT cells transfected as described in (a). Lower panel; hCAP18 transfected HaCaT cells demonstrate significantly higher proliferation rate (evaluated with 3H-Thymidine incorporation) compared with control cells.

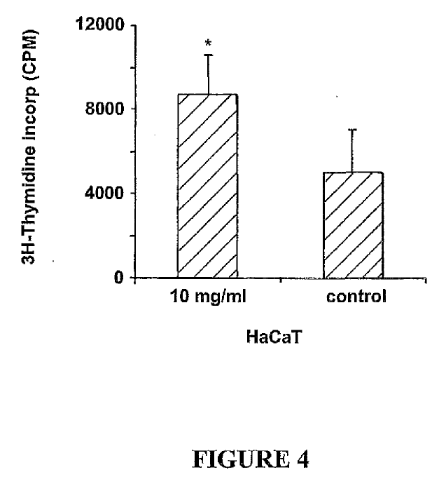

FIG. 4—Treatment with synthetic LL-37 peptide increases cell proliferation of epithelial cells.

HaCaT cells synchronized by serum starvation for 72 hours and then treated for 36 hours with 10 μg/ml of synthetic, biologically active LL-37 peptide (in DMEM+5% FCS+PEST) show significantly increased cell proliferation compared with non treated (control) HaCaT cells. Proliferation rate evaluated with [³H]-Thymidine incorporation.

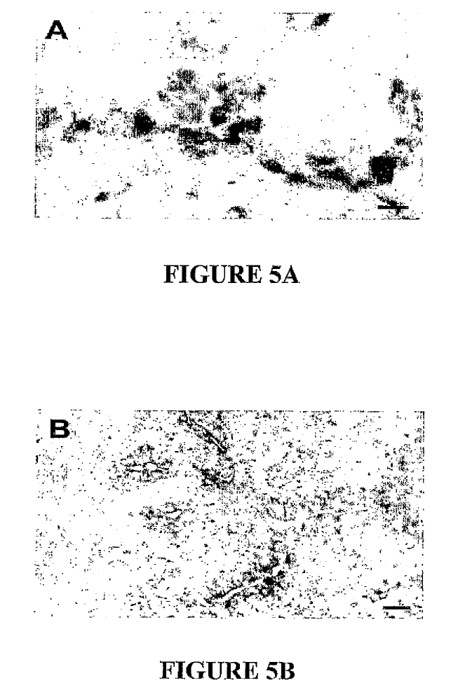

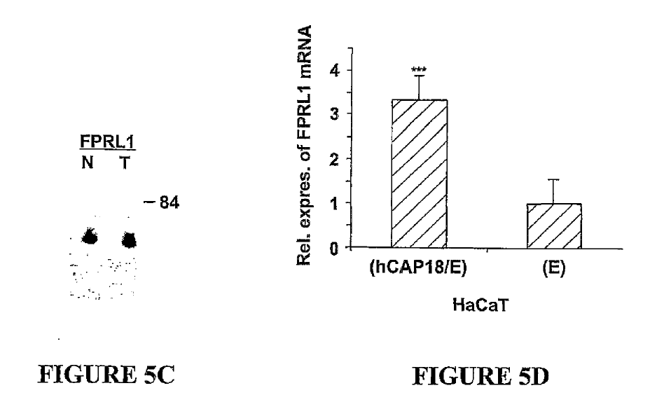

FIG. 5—The LL-37 receptor FPRL1 is expressed in breast cancer and in normal mammary gland epithelium.

(a) Section of ductal breast carcinoma Elston grade 2 (patient no 12, Table 2) with prominent immunoreactivity for FPRL1 receptor in tumour cells (red precipitate). (b) Section of normal mammary gland epithelium demonstrating immunoreactivity for FPRL1 in the ductal region (red precipitate). Photomicrographs show results obtained with the FPRL1 antiserum at 1:400 dilution. Scale bars (a)=50 μm; (b)=10 μm. (c) Immunoblotting revealed that the LL-37 receptor, FPRL1, was expressed in both normal (N) and breast cancer (T) tissue. (d) HaCaT transfected with a bicistronic vector hCAP18+EGFP (hCAP18/E) show significantly increased expression of FPRL1 receptor mRNA by real time PCR. HaCaT cells transfected with only EGFP (E) served as control.

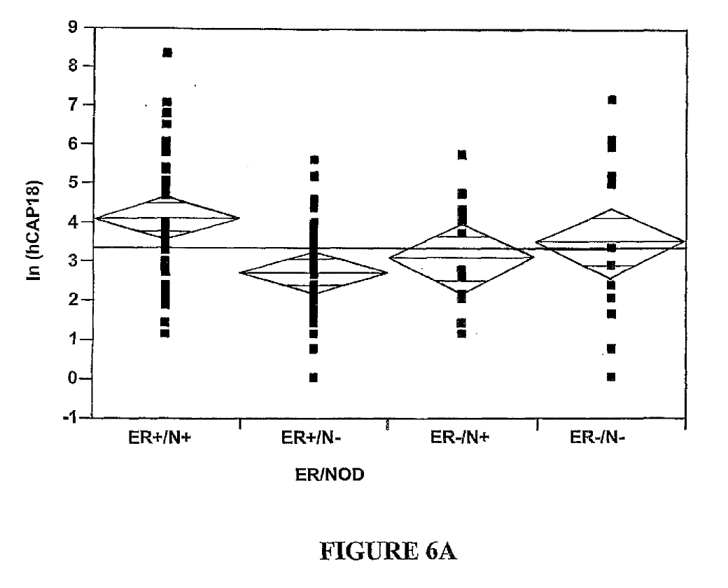

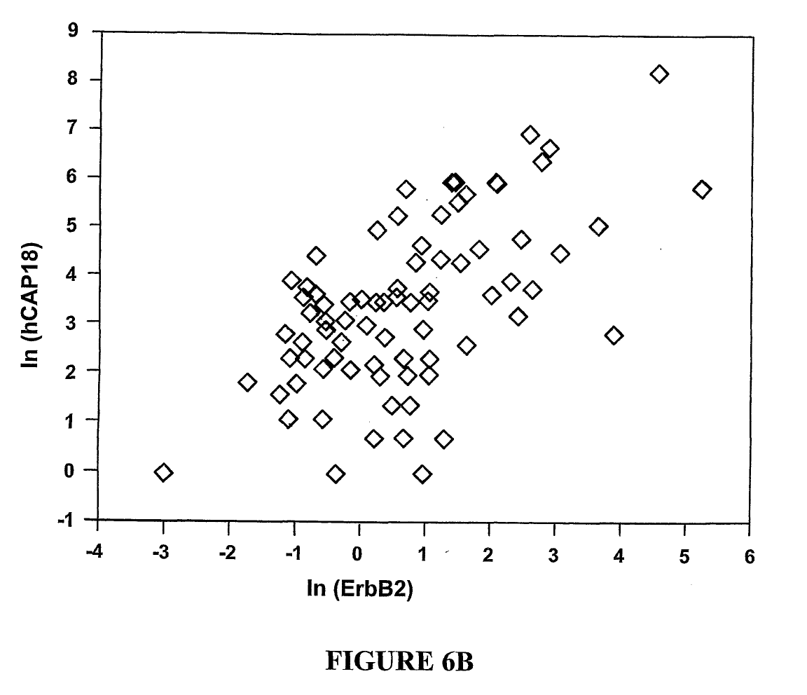

FIG. 6—Increased expression of hCAP18/LL-37 (displayed in logarithmic scale) in estrogen receptor (ER) and lymph node (N) positive breast tumours.

RNA was extracted from 140 breast tumours and from 4 unaffected breast tissue samples and reverse transcribed using random hexamers as primers. The expression of hCAP18 transcripts was determined by real-time PCR using 10 ng of cDNA according to standard protocols. The samples were normalized by quantification of 18S-RNA. The mean expression of the unaffected samples was arbitrarily set to 1. Mean and deviation are evaluated by Anova statistics.

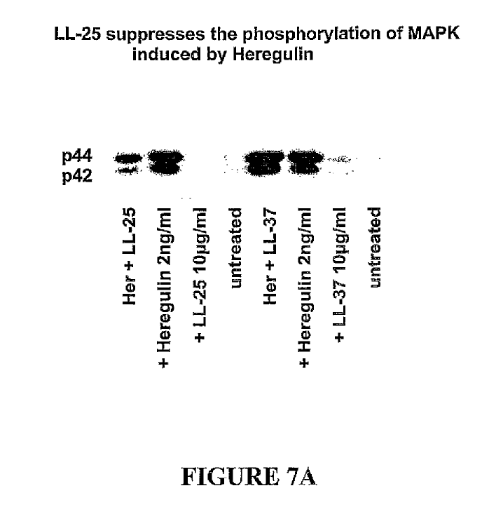

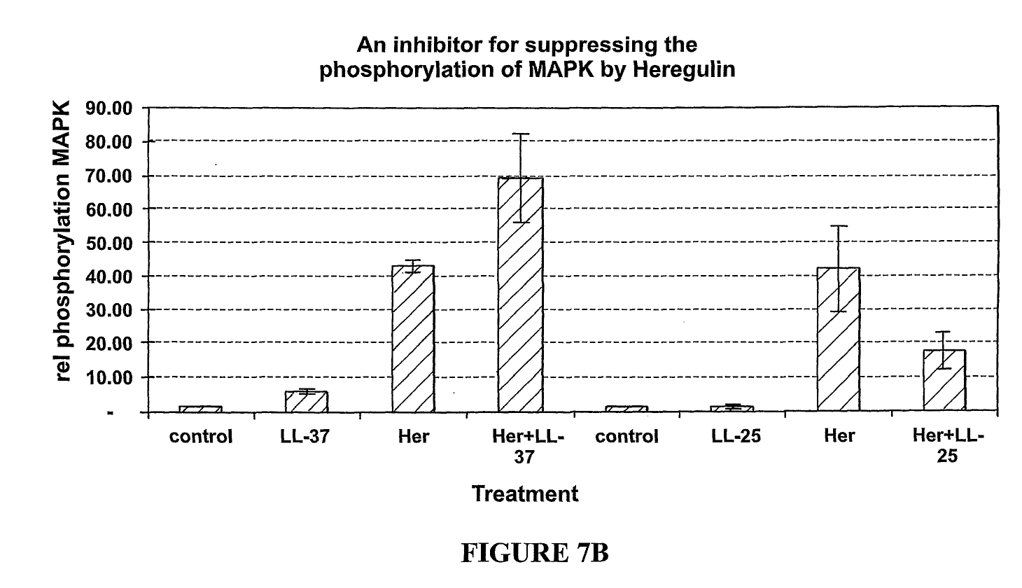

FIG. 7—LL-25 inhibits MAPK phosphorylation through Heregulin in the LL-37 expressing breast cancer line ZR75-1.

ZR75-1 cells were treated for 20 min with Heregulin (2 ng/ml), LL-25 or LL-37 or in combination as indicated. Protein extracts were analysed by Western blot analysis against phosphorylated MAPK. FIG. 7a shows the Western blot and FIG. 7b the quantitative evaluation of a triplicate of Western blot experiments. The study demonstrates that activity of endogenous LL-37 can be suppressed.

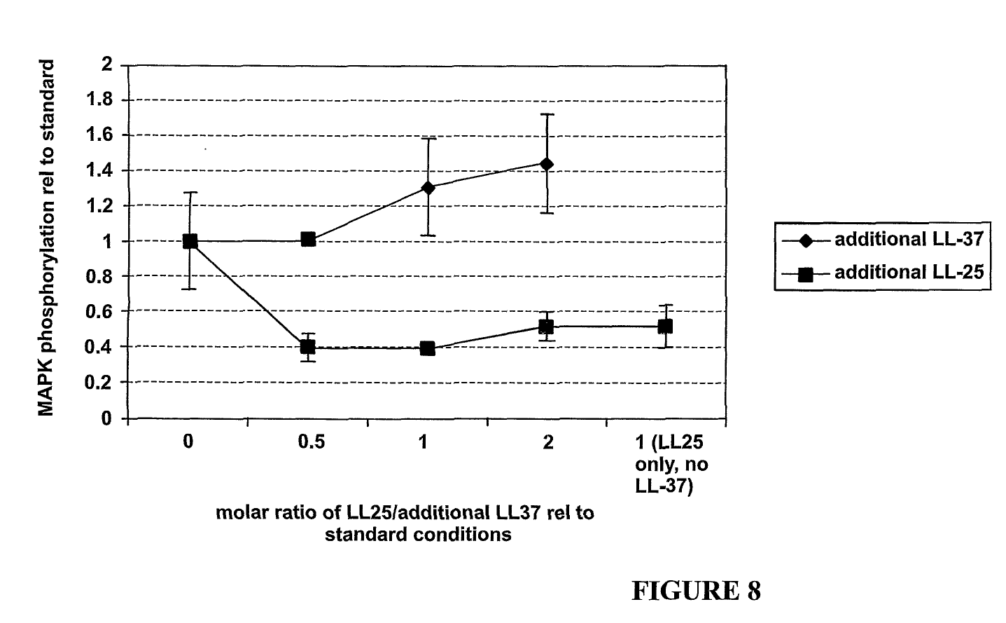

FIG. 8—Competitive inhibition of LL-37 by LL-25 on Heregulin-induced MAPK phosphorylation.

Heregulin (2 ng/ml) was added to the breast cancer line MCF7, which produces virtually no LL-37 on its own, together with LL-37 (2 μM) and LL-25 at concentrations as indicated. In the control experiments, the solvent (PBS), or LL-37, LL-25 or heregulin (HRG) were added as the only substrates. The quantitative evaluation of triplicate measurements is shown together with the Western blot of one of the triplicates. The results demonstrate that LL-25 is a competitive and highly efficient inhibitor to LL-37, even at 10% of the concentration of LL-37. The data also show that the endogenous effect of LL-37, even at low production, crucially contributes to MAPK activation through HRG, and can significantly be blocked by LL-25.

### EXAMPLES

### Example A

### The Antimicrobial Protein hCAP18/LL-37 is Highly Expressed in Breast Cancer and is a Putative Growth Factor for Epithelial Cells

### Introduction

In this example, the expression pattern of hCAP18/LL-37 in a series of breast carcinomas is investigated, demonstrating a marked upregulation of hCAP18 mRNA and protein in the tumour cells but not in the adjacent stroma. Interestingly, the highest levels of hCAP18 protein were detected among tumours with the highest histologic grade, whereas hCAP18 levels in some low grade tumours equalled those detected in the normal breast tissue. These findings clearly contrast with the hypothesised antitumour effect that has been proposed for antimicrobial peptides, but are consistent with recent findings which suggest a role for hCAP18/LL-37 in epithelial repair and angiogenesis ^{5, 10}. Further supporting hCAP18/LL-37 as a growth promoting factor, we here demonstrate that proliferation of epithelial cells was significantly enhanced both by treatment with synthetic biologically active LL-37 peptide and by transgenic expression of hCAP18.

### Material and Methods

### Tissues

Frozen tumour tissue from 28 breast cancer patients was obtained from the Department of Pathology, Danderyd Hospital, Stockholm, Sweden (Table 2). The tumours were scored according to Elston and Ellis I-III, following established guidelines 13. Cyclin A was used as proliferation marker (Nova-Castra Laboratories, Newcastle upon Tyne, UK). Estrogen receptor status was assessed on routinely processed paraffin sections. Uninvolved mammary tissue from eight patients with breast cancer and from two healthy individuals undergoing reductive breast surgery served as controls. All samples were examined by the same pathologist (B.S.) and classified as normal (Table 2). Written, informed consent was given by all patients. The study was approved by the Regional Committee of Ethics.

In Situ Hybridisation for hCAP18

A 435-bp hCAP18 full-length cDNA subcloned into pBluescript was used to in vitro transcribe [³⁵S]-labelled antisense and sense probes and in situ to hybridization was performed essentially as described ⁸ on samples 0-17 (Table 2).

### Immunohistochemistry

Immunohistochemistry was performed on samples 0-17 (Table 2). Cathelin-affinity-purified rabbit antiserum against recombinant hCAP18 ¹⁴ was used at 1:500 dilution as earlier described ¹⁰ according to the indirect peroxidase method using a Vectastain kit (Vector Laboratories, Burlingame, USA). To ascertain the specificity of the staining, immunoabsorption was performed as earlier reported ¹⁰. For detection of the FPRL1 receptor, affinity-purified goat polyclonal antibody was used at 1:400 dilution (Santa Cruz Biotechnology, Santa Cruz, Calif.) according to the indirect peroxidase method.

### Protein Extraction and Western Blot Analysis

Frozen tumour tissues, 16-60 mg, were homogenised in lysine buffer using an electric homogeniser. Proteins from tumour tissues and cell lines were extracted in SDS-containing sample buffer according to standard protocols ¹⁵. The protein concentration was determined by a spectrophotometric assay and adjusted with SDS-containing sample buffer to equal protein concentration ¹⁶ For the detection of hCAP18/LL-37 the extracts were separated on 16.5% Tris-Tricine Ready gels (Bio-Rad Laboratories, Hercules, Calif.). Recombinant cathelin ¹⁷ and synthetic LL-37 peptide were used as size references. For the detection of ERK1/2 and FPRL1, protein was separated on 12% and 8% Tris-Glycine gels respectively. To confirm that approximately equal amounts of protein in each sample were blotted, the filters were reversibly stained with a 3% Ponceau S solution (Sigma Aldrich, USA) in 3% TCA, before incubating with the primary antibody. Affinity purified anti-cathelin antiserum 17, affinity-purified anti-LL-37 antiserum ¹⁰, anti-FPRL1 antiserum (sc18191, Santa Cruz Biotechnology, CA) and monoclonal anti-ERK1/2 antibody (Cell Signaling Technology, Beverly Mass.) were all used at 1:1000 dilution. After electroblotting onto nitrocellulose filters (Schleicher & Schuell, Dassel, Germany), and sequential incubation with primary antibodies and horse-radish-peroxidase conjugated IgG (Santa Cruz Biotechnology, Santa Cruz, Calif.), signals from enhanced chemiluminiscence (ECL, Amersham Biosciences, Piscataway, N.J.) were captured with a CCD camera (LAS 1000, Fujifilm, Tokyo, Japan).

### ELISA

A sandwich ELISA previously described ¹⁷ was used to quantify hCAP18 in protein extracts from normal mammary gland and tumour tissues.

Expression analysis of hCAP18 by Real-Time PCR

RNA from four normal samples and four tumours was extracted with the Qiagen RNeasy kit (Operon Biotechnologies, Cologne, Germany) and reverse transcribed with a first strand synthesis kit (Amersham Biosciences, Norwalk, Conn.). RNA was quantified by Real-Time PCR on an ABI Prism 7700 (Applied Biosystems) using 10 ng of cDNA according to standard protocols. The samples were evaluated in triplicates. Sequences were 5′-GTCACCAGAGGATTGTGACTTCAA-3′ [SEQ ID NO:2] and 5′-TTGAGGGTCACTGTCCCCATA-3′ [SEQ ID NO:3] for the primers, and 6-FAM-5′-CCGCTTCACCAGCCCGTCCTT-3′-BHQ1 [SEQ ID NO:4] for the fluorigenic probe. The samples were normalised by quantification of 18S-RNA (Assay on Demand, Applied Biosystems). The mean expression of the normal samples was arbitrarily set to 1.

### Synthetic LL-37 Peptide

LL-37 peptide was synthesised and purified by HPLC to a purity of 98% (PolyPeptide Laboratories A/S, Hillerød, Denmark). Biological activity of the peptide was confirmed in an antibacterial assay ¹⁸.

### LL-37 Peptide Treatment of Epithelial Cells

A spontaneously immortalised human keratinocyte cell line (HaCaT) ¹⁹ was cultured in DMEM (Dulbecco's modified Eagle's medium, Gibco BRL, Life Technologies, Scotland) supplemented with 10% FCS (fetal calf serum, Hy-Clone, Boule Nordic AB Huddinge, Sweden) and antibiotics (PEST=penicillin 50 U/l and streptomycin 50 mg/ml, Gibco BRL). Cells were harvested at 70% confluence and seeded in 96-well plates, 2000 cells in 100 μl medium (DMEM+10% FCS and PEST). After 12 hours, medium was changed to serum free medium (DMEM+PEST) and cells were synchronized in G0/G1 by serum starvation for 72 hours and then treated with 100 μl of medium (DMEM+5% FCS+PEST) containing synthetic biologically active LL-37 peptide at 10 μg per ml. Cells treated with only DMEM+5% FCS and PEST served as control. The experiments were performed in quadruplicates. Cell proliferation was evaluated by [³H]-Thymidine incorporation. Cells were treated with 1 μCi/well of [³H]-Thymidine (20.00 Ci/mmol, Perkin Elmer Life Sciences Inc. Boston, Mass.) during 12 hours and harvested (Harvester 96, Tomtec, Orage, Conn.) onto a glass fiber filter (Wallac Oy Turku, Finland). The incorporation of [³H]-Thymidine was determined using a liquid scintillation counter (Microbeta Pluss, Wallac Sveriges AB). The experiment was repeated twice in 6 replicates.

Transgenic Expression of hCAP18 in HEK293 and HaCaT Cells

A Bfa1 fragment from Image clone 3057931 ²⁰ containing the entire coding sequence including the 16 bp of the 5′-untranslated region, was subcloned into the Smal-site of the bycistronic vector pIRES2-EGFP (BD Biosciences, Bedford, Mass.). HEK293 and HaCaT cells were transfected using Fugene (Roche Diagnostics, Indianapolis, Ind.) under standard conditions, and selected for two weeks with 400 ng/ml G418 (Invitrogen, Paisley, UK). Cells were sorted for EGFP expression with a MoFlo® high speed cell sorting flow cytometer (DakoCytomation, Fort Collins, Colo.) using Summit™ software for data analysis, and their expression of CAP18 was quantified by immunoblotting. Control cell lines were similarly established by transfection with the vector expressing EGFP only. The cell lines maintained a stable expression of CAP18 during several months of continued cultivation without any selection. The experiment was repeated twice in 30 replicates.

Proliferation Assays for HEK293 and HaCaT Cells Stably Transfected with hCAP18

HEK293 cells transfected with HCAP18 were harvested at 70% confluence and seeded in 24-well plates. After 24 hours, medium was changed and cells were cultured in 2 ml of medium (Optimem, Gibco BRL, Life Technologies, Scotland) supplemented with 5% FCS and PEST. Cells were harvested at day 6 and counted by flow cytometry (Becton Dickinson, Bedford, Mass.). Cell viability was measured with Trypan Blue; under all conditions less than 5% of the cells were Trypan Blue positive. All conditions were performed in triplicates. HEK293 cells transfected with the vector expressing only GFP served as control.

HaCaT cells transfected with hCAP18 were harvested at 70% confluence and seeded at 2000 cell per well in 96 well plates in DMEM with 10% FCS+PEST. Medium was changed 12 hours later to DMEM supplemented with 5% FCS+PEST. After 24 hours of culture, the cells were treated 12 hours with 1 μCi/well of [³H]-Thymidine, harvested and analysed as described above. HaCaT cells transfected with the vector only expressing EGFP served as control.

### Expression Analysis of FPRL1

RNA from HaCaT cells was extracted with the RNeasy kit (Qiagen) and reverse transcribed with a first strand synthesis kit (Amersham-Pharmacia). FPRL1 RNA was quantified by Real-Time PCR and normalized against 18S-RNA as described above. Sequences were 5′-TCTGCTGGCTACACTGTTCTGC-3′ [SEQ ID NO:2] and 5′-GACCCCGAGGACAAAGGTG-3′ [SEQ ID NO:3] for the primers, and 6-FAM 5′-CCCAAGCACCACCAATGGGAGGA-3′-BHQ1 [SEQ ID NO:4] for the fluorigenic probe.

### Pertussis Toxin Assay

To assess the involvement of FPRL1 in mediating the stimulation of epithelial cell proliferation induced by hCAP18/LL-37, HaCaT cells were treated with the G-protein-coupled receptor inhibitor pertussis toxin. Cells were preincubated with pertussis toxin (Sigma-Aldrich, Switzerland) 24 h before the LL-37 treatment in a final toxin concentration of 20 ng/ml. Medium was changed 48 hours after cell seeding and the HaCaT cells were treated with 100 μl of medium (DMEM+5% FCS and PEST) containing synthetic biologically active LL-37 peptide at 5 or 10 μg per ml respectively. Cells treated with only DMEM+5% FCS and PEST served as control.

### Assay of Phosphorylated ERK1/2 in LL-37 Treated HaCaT Cells

HaCaT cells were seeded at 10% confluence and kept in DMEM with 0.2% FCS for 36 hours. For the next 48 hours, cells were cultured in DMEM with 1% or 5% FCS respectively, and in presence or absence of LL-37 at 10 μg/ml, with daily changes of medium. EGF at 10 ng/ml served as positive control. The expression of phosphorylated ERK 1/2 was evaluated by Western blot analysis with a mouse monoclonal antibody (Cell Signaling Technology, Beverly Mass.).

### Statistical Analysis

Values are presented as mean number of cells or counts per minute (CPM) plus or minus SD. Comparisons between groups were analysed by two-sided t-tests. Results were considered statistically significant for P values <0.05. For the analysis of the expression in tumours, a one-tailed t-test was performed on hCAP18 protein levels at a significance level of <0.05.

### Results

hCAP18/LL-37 is Expressed in Breast Cancer

Patient details are presented in table 2.

By in situ hybridisation, there was low signal for hCAP18 mRNA (not shown) and weak immunoreactivity for HCAP18 protein in breast tissue from a healthy control (FIG. 1g) and in uninvolved breast cancer (not shown). All breast cancer tissues showed immunoreactivity for hCAP18 in the tumour cells and not in the stroma (FIG. 1a, c, d). The tumour cell population was not homogenous with regard to hCAP18 immunoreactivity, strongly positive cells being found adjacent to cells devoid of detectable hCAP18 (FIG. 1c). Immunoabsorption with cathelin recombinant protein abolished the hCAP18 immunoreactivity (FIG. 1e, f). By in situ hybridisation, positive signal for hCAP18 mRNA was detected in the same areas closely matching the expression pattern obtained with immunohistochemistry (FIG. 1b). Signal intensity varied and was most prominent among high grade tumours. Control sections hybridised with the sense hCAP18 cRNA probe lacked specific signal for hCAP18 mRNA (not shown).

Quantification of hCAP18 protein by ELISA in breast cancer tissue extracts revealed no difference between Elston I and II grade tumours, but clearly higher hCAP18 levels in tumours of the highest malignancy grade (Table 2). The difference between Elston III grade and the remaining tumours was statistically significant p<0.01). Ten of the 13 grade III tumours reached or exceeded a hCAP18 concentration of 5 ng/mg total protein. Only 2 of the remaining 18 tumour samples reached this level. We also assayed four specimen of healthy breast tissue which revealed similar levels as Elston I or II tumours. To verify the expression pattern obtained by ELISA, we performed Real-Time PCR on four normal samples and on four of the tumours. The results of transcript quantification were in line with the data on protein expression (Table 2).

By iminunoblotting, all tumours and normal breast tissues investigated showed immunoreactive bands corresponding to the intact non-processed 18 kDa holoprotein (FIG. 2). In 4 of the 5 investigated grade III tumours (Table 2, sample 6-10), we also detected bands corresponding to LL-37, the processed hCAP18 protein (FIG. 2). The antiserum used is raised against the hCAP18 holoprotein and detects LL-37 at high concentrations even though it is affinity purified against the cathelin peptide ¹⁰.

hCAP18/LL-37 Increases Proliferation of Epithelial Cells

HEK293 and HaCaT cells transfected with a hCAP18 (hCAP18/E) expression vector demonstrated significantly higher proliferation rate than control cells transfected with the vector expressing EGFP only (E) (FIGS. 3 A and B). By immunoblotting of protein extracts from the transfected HEK293 and HaCaT cells, we confirmed that these hCAP18 vector-containing cells produced the holoprotein (FIGS. 3 A and B) and a 4 kD immunoreactive band corresponding to LL-37 was detected in the cell medium (data not shown). In addition, HaCaT cells cultured at 5% fetal calf serum and treated with synthetic biologically active LL-37 peptide at 10 μg/ml demonstrated a significant increase in cell proliferation (FIG. 4).

TABLE 2

| Sample^{a} | Age | | | | | | hCAP18^{f} | Real Time | | Axillar | Clinical |
|---|---|---|---|---|---|---|---|---|---|---|---|
| (no) | (year) | Type | Grading^{b} | ER^{c} | Cyclin A^{d} | IH & ISH^{e} | (ng/mg) | PCR^{g} | Treatment^{h} | LNⁱ | Status^{j} |
| 0 | 30 | Healthy | | | | • | | | | | |
| 1 | 72 | Healthy | | | | • | 0.7 | | | | |
| 2 | 53 | Lobular | I | + | L | • | 2.3 | | M, TAM | − | 0 |
| 3 | 65 | Ductal | II | + | H | • | 1.1 | | M, CT | − | CIS |
| 3b | | Normal | | | | • | | | | | |
| 4 | 37 | Ductal | I | + | H | • | 2.3 | | PM, Rx, TAM | − | 0 |
| 5 | 69 | Colloid | I | + | L | • | 1.7 | | PM, Rx, TAM | − | 0 |
| 6 | 84 | Ductal | III | − | H | • | 5.4 | | M | − | † |
| 7 | 53 | Ductal | III | − | H | • | 35.8 | | M, Rx, CT | + | 0 |
| 8 | 55 | Ductal | III | − | H | • | 5 | 8 | PM, Rx, CT | + | Metastasis |
| 9 | 73 | Ductal | III | − | H | • | 11.8 | | M, TAM | − | † |
| 10 | 47 | Ductal | III | − | H | • | 5.3 | | M, CT, RX | − | † |
| 11 | 64 | Ductal | II | + | H | • | 1.6 | | M, Rx, TAM | − | 0 |
| 12 | 52 | Ductal | II | + | H | • | 5 | | PM, Rx, CT | + | 0 |
| 12b | | Normal | | | | • | | | | | |
| 13 | 69 | Ductal | I | + | L | • | 0.9 | | PM, Rx, TAM | + | 0 |
| 14 | 31 | Ductal | II | + | L | • | 4 | | M, Rx, CT | + | 0 |
| 14b | | Normal | | | | | | | | | |
| 15 | 58 | Ductal | I | + | L | • | 3.9 | | PM, Rx, TAM | − | 0 |
| 16 | 70 Right | Ductal | I | + | L | • | 4.12 | | PM, Rx, TAM | − | 0 |
| 16 | Left | Lobular | II | + | L | • | 4.56 | | M, TAM | − | 0 |
| 16b | | Normal | | | | • | | | | | |
| 17 | 70 | Tubular | I | + | L | • | | | M, Rx, TAM | − | 0 |
| 18 | 76 | Ductal | I | + | L | | 3.9 | | M, Rx, CT | + | 0 |
| 19 | 64 | Ductal | III | + | H | | 3.9 | | PM, Rx, CT | − | 0 |
| 20 | 69 | Ductal | I | + | L | | 4.7 | | PM, Rx, TAM | − | 0 |
| 21 | 78 | Lobular | III | + | H | | 38 | | M, CT | + | 0 |
| 22 | 67 | Ductal | III | + | H | | 4.0 | | M, CT | nd | 0 |
| 23 | 82 | Colloid | I | + | L | | 11.7 | | M, CT, | nd | 0 |
| 24 | 76 | Ductal | II | − | L | | 3.7 | | M, Rx, CT | − | 0 |
| 25 | 44 | Ductal | III | + | H | | 7.0 | 11 | M, Rx, CT | + | 0 |
| 26 | 79 | Medullary | III | + | H | | 4.8 | 18 | M, Rx, CT | + | 0 |
| 27 | 66 | Ductal | I | + | L | | 8.7 | | PM, Rx, CT | − | † |
| 28 | 58 | Ductal | III | − | H | | 41 | 11 | M, Rx, CT | + | † |
| 29 | 65 | Metastasis | — | + | H | | 29.5 | | CT | + | Metastasis |
| 30 | 54 | Lobular | III | + | H | | 5.8 | | M, Rx, CT | + | 0 |
| 31 | 81 | Normal | | | | | 1.2 | 0.6 | | | |
| 32 | 60 | Normal | | | | | 2.9 | 1.1 | | | |
| 33 | 65 | Normal | | | | | 2.9 | 1.5 | | | |
| 34 | 55 | Normal | | | | | | 1.1 | | | |

^{a}Tissues from 28 patients with breast carcinoma, normal mammary tissue from 8 patients with breast carcinoma and from 2 healthy individuals undergoing reconstructive breast surgery (sample no 0 and 1).
^{b}Tumours graded according to Elston and Ellis.
^{c}Assessment of estrogen receptor (ER) status performed with immunohistochemistry.
^{d}Percentage of cells expressing proliferation marker Cyclin A. Low (L) < 5%, High (H) ≧ 5%.
^{e}Tissues investigated (•) with immunohistochemistry (IH) and in situ hybridisation (ISH) for hCAP18.
^{f}Protein extraction from tissues, hCAP18 levels measured with ELISA and presented as ng hCAP18 per mg total protein.
^{g}RNA extraction from tissues, hCAP18 mRNA measured with Real Time PCR (TaqMan), the mean of normal arbitrarily set as one.
^{h}M = mastectomy, PM = partial mastectomy, Rx = radiation, CT = chemo therapy, TAM = tamoxifene.
ⁱAxillary lymph nodes status at surgery. nd = not done.
^{j}Clinical status was assessed 1.5-2 years after diagnosis.
† = dead,
0 = no clinical relapse,
CIS = carcinoma in situ.

### The LL-37 Receptor FPRL1 is Expressed in Breast Cancer

The G-protein-coupled receptor, FPRL1 has been shown to mediate LL-37 induced effects in eukaryote cells ^{4, 5} and to assess its potential role in the present setting, we investigated the expression of FPRL1 protein in mammary tissue and found strong immunoreactivity for FPRL1 both in breast cancer cells and in normal glandular epithelium (FIG. 5a,b). Immunoblotting confirmed that FPRL1 was expressed in both tissues (FIG. 5c). In addition, transgenic expression of hCAP18 significantly increased the expression of FPRL1 mRNA (FIG. 5d) in HaCaT cells which may further support the involvement of FPRL1 in hCAP18/LL-37 signalling. However, pretreatment of HaCat cells with pertussis toxin did not abolish but suppressed the proliferation of these cells by approximately 50% (not shown), indicating that FPRL1 may not be uniquely involved in mediating hCAP18/LL-37 growth stimulatory effects in these cells. To test the possible involvement of ERK1/2 in activation of epithelial cell proliferation, we treated HaCaT cells with synthetic biologically active LL-37 but there was no significant activation of ERK1/2, which indicates that EGFR is not involved in mediating the LL-37 stimulatory effect on HaCaT cell proliferation.

### Discussion

In the present study we demonstrate that hCAP18/LL-37 is constitutively produced in normal mammary gland epithelium. This is consistent with a role for LL-37 in antimicrobial barrier protection in human and agrees with earlier reports where low constitutive expression of LL-37 was found in normal quiescent epithelium, in contrast to the pronounced expression seen in association with injury and inflammation ⁷⁻¹⁰. Constitutive expression of antimicrobial peptides has previously been detected in various exocrine glands such as the human cathelicidin LL-37 in sweat glands, the cathelicidin CRAMP in murine salivary glands and beta-defensins in human salivary glands ²¹⁻²³ Expression of human beta-defensin-2 (hBD-2) mRNA in mammary glands was reported by Bals et al in 1998 and recently other groups have found constitutive hBD-1 expression in mammary glandular tissue of non-lactating women as well as in breast tissue during lactation and in breast milk ²⁴⁻²⁶.

Interestingly, the production of hCAP18 was most notably increased in the breast epithelium of high-grade tumours compared with normal mammary epithelium or low-grade tumours. The hCAP18 expression was however neither universal nor uniform, i.e. not all cancer cells were positive for hCAP18, but distinctly positive cells were found adjacent to cells devoid of detectable hCAP18 mRNA and protein (FIG. 1c), and the degree of expression varied considerably among cells in all tumour types. This may reflect a complex yet strictly controlled regulation of hCAP18 as has been suggested for human alpha-defensins in renal cell carcinoma ²⁷.

In our study, the highest hCAP18/LL-37 levels were detected among tumours with the highest histologic grade. Although the difference in hCAP11 expression between high grade tumours on the one hand and low grade and normal breast tissues is statistically significant, there is no strict correlation. Within all groups there were tumours expressing hCAP18 at the level of the healthy samples and two of the grade I tumours showed a relatively high expression otherwise only observed among the grade III tumours. However, given the limitations by the sample numbers, our observations suggest a potential correlation between degree of malignancy and expression of hCAP18/LL-37. One may argue that the overexpression of hCAP18 in breast cancer may result from failures in intracellular pathways affecting the regulation of hCAP18, and that hCAP18 expression reflects these alterations rather than providing a growth advantage for the tumour. However, coupled with the in vitro studies presented here, we believe that the data underline the potential role for LL-37 in promoting tumour growth.

The biological role of antimicrobial peptides in carcinomas is unclear. High hBD-2 protein concentration and marked immunoreactivity for both human alpha- and beta-defensins have been found in various oral carcinomas and it has been suggested that the increased levels of these antimicrobial peptides may be the result of infection and/or stimulation by cytokines ²⁸⁻³⁰, Other studies have proposed that antimicrobial peptides isolated from insects, e.g. melittin and cecropin related peptides exert antitumour effects on mammalian tumour cells ³¹⁻³⁴. Moreover, vector mediated delivery and expression of the coding sequences for cecropin and mellitin in a human bladder carcinoma cell line suppressed tumourigenicity in nude mice ¹¹. Likewise, transgenic expression of the porcine cathelicidin PR-39, reduced the invasive capacity of human hepatocellular carcinoma ¹².

Although further studies are required to elucidate the functions of antimicrobial peptides in cancer, a multifunctional role for these peptides is becoming increasingly manifest. In addition to pathogen inactivation through a direct membrane effect, LL-37 exerts chemotactic effects in vitro, inducing migration of human neutrophils, monocytes, subsets of T-cells and mast cells ^{4, 35, 36}. This chemotactic activity is dependent on binding of LL-37 to FPRL1, a pertussis toxin-sensitive, membrane bound G-protein-coupled receptor ⁴. Additional suggested functions for hCAP18/LL-37 include a role in epithelial repair and angiogenesis by promoting re-epithelialization of skin wounds and neovascularization ^{5, 10}.

Thus, the marked hCAP18/LL-37 expression in breast cancer cells presented herein may reflect a growth advantage for these tumour cells. To test this hypothesis, we transfected the human epithelial cell lines HEK293 and HaCaT with an hCAP18 expression vector and found a significant increase in proliferation of transfected cells. In addition, synthetic biologically active LL-37 peptide significantly increased proliferation of HaCaT cells. These findings clearly contrast with the suggested antitumour effect proposed for antimicrobial peptides, but are consistent with recent findings by Müller et al, that human alfa-defensins may modulate progression of renal cell carcinoma (RCC). These defensins were found in tumour cells of RCC as well as in normal tubular epithelial of the kidney and at physiological concentrations stimulated tumour cell proliferation ²⁷.

Our in vitro studies suggest that LL-37 stimulates proliferation of epithelial cells, partially through FPRL1 since blocking the receptor with pertussis toxin decreased the exogenous LL-37 proliferation effect by approximately 50%, possibly indicating the involvement also of other receptors. In a recent study it was suggested that LL-37 activates airway epithelial cells by activation of the mitogen-activated protein kinase/extracellular signal-regulated kinase (MAPK/ERK kinase=MEK) via transactivation of the epidermal growth factor receptor (EGFR) ³⁷. However, in our experiments we did not detect any significant activation of ERK1/2.

In conclusion, the results presented herein indicates that LL-37 promotes tumour growth.

### REFERENCES

1. Gudmundsson G H, Agerberth B, Odeberg J, Bergman T, Olsson B, Salcedo R. The human gene FALL39 and processing of the cathelin precursor to the antibacterial peptide LL-37 in granulocytes. Eur J Biochem 1996; 238:325-32.
2. Zanetti M, Gennaro R, Romeo D. Cathelicidins: a novel protein family with a common proregion and a variable C-terminal antimicrobial domain. FEBS Lett 1995; 374:1-5.
3. Agerberth B, Gunne H, Odeberg J, Kogner P, Boman H G, Gudmundsson G H. FALL-39, a putative human peptide antibiotic, is cysteine-free and expressed in bone marrow and testis. Proc Natl Acad Sci USA 1995; 92: 195-99.
4. Yang D, Chen Q, Schmidt A P, Anderson G M, Wang J M, Wooters J, Oppenheim J J, Chertov O. LL-37, the neutrophil granule- and epithelial cell-derived cathelicidin, utilizes formyl peptide receptor-like 1 (FPRL1) as a receptor to chemoattract human peripheral blood neutrophils, monocytes, and T cells. J Exp Med 2000; 192:1069-74.
5. Koczulla R, von Degenfeld U, Kupatt C, Krotz F, Zahler S, Gloe T, Issbrucker K, Unterberger P, Zaiou M, Lebherz C, Karl A, Raake P, et al. An angiogenic role for the human peptide antibiotic LL-37/hCAP-18. J Clin Invest 2003; 111:1665-72,
6. Cowland J B, Johnsen A H, Borregaard N. hCAP-18, a cathelin/pro-bactenecin-like protein of human neutrophil specific granules. FEBS Lett 1995; 368:173-76.
7. Frohm M, Agerberth B, Ahangari G, Ståhle-Bäckdahl M, Lidén S, Wigzell H, Gudmundsson G H. The expression of the gene coding for the antibacterial peptide LL-37 is induced in human keratinocytes during inflammatory disorders. J Biol Chem 1997; 272:15258-63.
8. Frohm Nilsson M, Sandstedt B, Sørensen O, Weber G, Borregaard N, Ståhle-Bäckdahl M. The human cationic antimicrobial protein (hCAP18), a peptide antibiotic, is widely expressed in human squamous epithelia and colocalizes with interleukin-6. Infect Immun 1999; 67:2561-66.
9. Dorschner R A, Pestonjamasp V K, Tamakuwala S, Ohtake T, Rudisill J, Nizet V, Agerberth B, Gudmundsson G H, Gallo R L. Cutaneous injury induces the release of cathelicidin anti-microbial peptides active against group A Streptococcus. J Invest Dermatol 2001; 117:91-97.
10. Heilborn J D, Nilsson M F, Kratz U, Weber G, Sorensen O, Borregaard N, Stahle-Backdahl M. The cathelicidin anti-microbial peptide LL-37 is involved in re-epithelialization of human skin wounds and is lacking in chronic ulcer epithelium. J Invest Dermatol 2003; 120:379-89.
11. Winder D, Gunzburg W H, Erfle V, Salmons B. Expression of antimicrobial peptides has an antitumour effect in human cells. Biochem Biophys Res Commun 1998; 242:608-12.
12. Ohtake T, Fujimoto Y, Ikuta K, Saito H, Ohhira M, Ono M, Kohgo Y. Proline-rich antimicrobial peptide, PR-39 gene transduction altered invasive activity and actin structure in human hepatocellular carcinoma cells. Br J Cancer 1999; 81:393-403.
13. Pathology NCGfBS. Pathology reporting in breast cancer screening. second edition, NHSBSP Publication 1995.
14. Sørensen O, Bratt T, Johnsen A H, Madsen M T, Borregaard N. The human antibacterial cathelicidin, hCAP-18, is bound to lipoproteins in plasma. J Biol Chem 1999; 274:22445-51.
15. Ausubel F M, Brent R, R. E. K, Moore D M, Seidman J G, Smith J A, Struhl K. Current Protocols in Molecular Biology: Wiley & Sons, Hoboken, N.J., 2003.
16. Schaffner W, Weissmann C. A rapid, sensitive, and specific method for the determination of protein in dilute solution. Anal Biochem 1973; 56:502-14.
17. Sørensen O, Cowland J B, Askaa J, Borregaard N. An ELISA for hCAP-18, the cathelicidin present in human neutrophils and plasma. J Immunol Methods 1997; 206:53-59.
18. Frohm M, Gunne H, Bergman A C, Agerberth B, Bergman T, Boman A, Lidén S, Jörnvall H, Boman H G. Biochemical and antibacterial analysis of human wound and blister fluid. Eur J Biochem 1996; 237:86-92.
19. Boukamp P, Petrussevska R T, Breitkreutz D, Hornung J, Markham A, Fusenig N E. Normal keratinization in a spontaneously immortalized aneuploid human keratinocyte cell line. J Cell Biol 1988; 106:761-71.
20. Lennon G, Auffray C, Polymeropoulos M, Soares M B. The I.M.A.G.E. Consortium: an integrated molecular analysis of genomes and their expression. Genomics 1996; 33:151-2.
21. Murakami M, Ohtake T, Dorschner R A, Gallo R L. Cathelicidin antimicrobial peptides are expressed in salivary glands and saliva. J Dent Res 2002; 81:845-50.
22. Murakami M, Ohtake T, Dorschner R A, Schittek B, Garbe C, Gallo R L. Cathelicidin anti-microbial peptide expression in sweat, an innate defense system for the skin. J Invest Dermatol 2002; 119:1090-5.
23. Sahasrabudhe K S, Kimball J R, Morton T H, Weinberg A, Dale B A. Expression of the antimicrobial peptide, human beta-defensin 1, in duct cells of minor salivary glands and detection in saliva. J Dent Res 2000; 79:1669-74.
24. Bals R, Wang X, Wu Z, Freeman T, Bafna V, Zasloff M, Wilson J M. Human beta-defensin 2 is a salt-sensitive peptide antibiotic expressed in human lung. J Clin Invest 1998; 102:874-80.
25. Tunzi C R, Harper P A, Bar-Oz B, Valore E V, Semple J L, Watson-MacDonell J, Ganz T, Ito S. Beta-defensin expression in human mammary gland epithelia. Pediatr Res 2000; 48:30-5.
26. Jia H P, Starner T, Ackermann M, Kirby P, Tack B F, McCray P B, Jr. Abundant human beta-defensin-1 expression in milk and mammary gland epithelium. J Pediatr 2001; 138:109-12.
27. Müller C A, Markovic-Lipkovski J, Klatt T, Gamper J, Schwarz G, Beck H, Deeg M, Kalbacher H, Widmann S, Wessels J T, Becker V, Muller G A, et al. Human alpha-defensins HNPs-1, -2, and -3 in renal cell carcinoma: influences on tumour cell proliferation. Am J Pathol 2002; 160:1311-24.
28. Mizukawa N, Sawaki K, Yamachika E, Fukunaga J, Ueno T, Takagi S, Sugahara T. Presence of human beta-defensin-2 in oral squamous cell carcinoma, Anticancer Res 2000; 20:2005-7.
29. Mizukawa N, Sawaki K, Nagatsuka H, Kamio M, Yamachika E, Fukunaga J, Ueno T, Takagi S, Sugahara T. Human alpha- and beta-defensin immunoreactivity in oral mucoepidermoid carcinomas. Anticancer Res 2001; 21:2171-4.
30. Sawaki K, Mizukawa N, Yamaai T, Yoshimoto T, Nakano M, Sugahara T. High concentration of beta-defensin-2 in oral squamous cell carcinoma. Anticancer Res 2002; 22:2103-7.
31. Jaynes J M, Julian G R, Jeffers G W, White K L, Enright F M. In vitro cytocidal effect of lytic peptides on several transformed mammalian cell lines. Pept Res 1989; 2:157-60.
32. Sharma S V. Melittin-induced hyperactivation of phospholipase A2 activity and calcium influx in ras-transformed cells. Oncogene 1993; 8:939-47.
33. Moore A J, Devine D A, Bibby M C. Preliminary experimental anticancer activity of cecropins. Pept Res 1994; 7:265-9.
34. Hui L, Leung K, Chen H M. The combined effects of antibacterial peptide cecropin A and anti-cancer agents on leukemia cells. Anticancer Res 2002; 22:2811-6.
35. Agerberth B, Charo J, Werr J, Olsson B, Idali F, Lindbom L, Kiessling R, Jörnvall H, Wigzell H, Gudmundsson G H. The human antimicrobial and chemotactic peptides LL-37 and alpha-defensins are expressed by specific lymphocyte and monocyte populations. Blood 2000; 96:3086-93.
36. Niyonsaba F, Twabuchi K, Someya A, Hirata M, Matsuda H, Ogawa H, Nagaoka I. A cathelicidin family of human antibacterial peptide LL-37 induces mast cell chemotaxis. Immunology 2002; 106:20-6.
37. Tjabringa G S, Aarbiou J, Ninaber D K, Drijfhout J W, Sorensen O E, Borregaard N, Rabe K F, Hiemstra P S. The antimicrobial peptide LL-37 activates innate immunity at the airway epithelial surface by transactivation of the epidermal growth factor receptor. J Immunol 2003; 171:6690-6.

### Example B

### Increased Expression of hCAP18/LL-37 in Estrogen Receptor (ER) and Lymph Node (N) Positive Breast Tumours

### Materials and Methods

RNA was extracted from 140 breast tumours and from 4 unaffected breast tissue samples and reverse transcribed using random hexamers as primers. The expression of hCAP18 transcripts was determined by real-time PCR using 10 ng of cDNA according to standard protocols (as described above).

### Results and Discussion

Results are shown in FIG. 6. The mean expression of the unaffected samples was arbitrarily set to 1. Mean and deviation were evaluated by Anova statistics.

The expression of hCAP18 is significantly higher (by about 5 times) in ER positive tumors when lymph nodes have developed, than without lymph nodes.

### Example C

### The Effect of LL-25 on Phosphorylation of MAPK

ZR75-1 ells were grown in Optimem and 10% FCS, and plated in 12-well plates at 100 000 cells/well, corresponding 70% confluency. After reattachment, cells were starved for 48 hours in DMEM, no FCS. Test substrate dissolved in 50 μl PBS were added to the medium, and cells were incubated for 20 min. The stimulation was stopped by washing the cells with ice-cold PBS containing 1 mM NaF, 100 μM Na₃VO₄, and 2 mM PMSF, and then lysed in 300 μl SDS lysis buffer containing the inhibitors as above. Proteins were separated in an 8% gel, and blotted to nitrocellulose filters according to standard conditions. Filters were reversibly stained with 3% Ponceau S, blocked with 4% NFDM in TBS/0.1% Tween20, and incubated o.n. with the primary antibody. For detection of phosphorylated MAPK 1/2 (pThr 202/pTyr 204), a monoclonal antibody (Cell Signaling Inc) was used at 1/2000. After washing and incubation with HRP.conjugated secondary antibody, chemoluminescence was induced by developing with ECL and ECL advanced at a ratio of 9:1. The signal was captured in a CCD camera (Fuji, Tokyo, Japan) for 1 min, and evaluated by Image Gauge Software. For quantification, the signals were normalized against the Ponceau S staining that had been scanned in and quantified using the same software.

FIG. 7a shows the Western blot and FIG. 7b the quantitative evaluation of a triplicate of Western blot experiments. The study demonstrates that activity of endogenous LL-37 can be suppressed.

In a further experiment, Heregulin (2 ng/ml) was added to the breast cancer line MCF7, which produces virtually no LL-37 on its own, together with exogenous LL-37 (2 μM) and LL-25 at concentrations as indicated. In the control experiments, the solvent (PBS), or LL-37, LL-25 or heregulin (HRG) were added as the only substrates. The quantitative evaluation of triplicate measurements is shown together with the Western blot of one of the triplicates. The results shown in FIG. 8 demonstrate that LL-25 is a competitive and highly efficient inhibitor of LL-37, even at 10% of the concentration of LL-37. The data also show that the endogenous effect of LL-37, even at low production, crucially contributes to MAPK activation through HRG, and can significantly be blocked by LL-25.

1-27. (canceled)

28. A method for treating cancer in a patient, the method comprising administering to the patient a polypeptide comprising an amino acid sequence according to SEQ ID NO: 1 or a biologically active fragment, variant, fusion or derivative thereof.

29. A method according to claim 28 wherein the patient is human.

30. A method according to claim 28 wherein the polypeptide, or fragment, variant, fusion or derivative thereof, is capable of inhibiting the proliferation of cancer cells.

31. A method according to claim 28 wherein the polypeptide, or fragment, variant, fusion or derivative thereof is capable of inhibiting metastasis of cancer cells.

32. A method according to claim 28 wherein the polypeptide, or fragment, variant, fusion or derivative thereof, is capable of inhibiting the proliferation and/or metastasis of cancer cells selectively.

33. A method according to claim 28 wherein the polypeptide, or fragment, variant, fusion or derivative thereof, is capable of inhibiting the proliferation of cancer cells by 20% or more compared to the proliferation of cancer cells which have not been exposed to the medicament, for example by at least 30%, 40%, 50%, 60%, 70%, 80%, 90% or more.

34. A method according to any claim 28 wherein the polypeptide, or fragment, variant, fusion or derivative thereof, is capable of inhibiting metastasis of cancer cells by 20% or more compared to metastasis of cancer cells which have not been exposed to the medicament, for example by at least 30%, 40%, 50%, 60%, 70%, 80%, 90% or more.

35. A method according to claim 28 wherein the cancer cells are epithelial cells.

36. A method according to claim 28 wherein the cancer cells are squamous cells.

37. A method according to claim 28 wherein the cancer cells are selected from the group consisting of cancer cells of the breast, bile duct, brain, colon, stomach, reproductive organs, lung and airways, skin, gallbladder, liver, nasopharynx, nerve cells, kidney, prostate, lymph glands and gastrointestinal tract.

38. A method according to claim 37 wherein the cancer cells are breast cancer cells.

39. A method according to claim 38 wherein the breast cancer cells are Elston grade III cells.

40. A method according to claim 38 wherein the breast cancer cells are metastatic.

41. A method according to claim 28 wherein the polypeptide comprises or consists of an amino acid sequence according to SEQ ID NO: 1.

42. A method according to claim 28 wherein the polypeptide consists of an amino acid sequence according to SEQ ID NO: 1

43. A method according to claim 28 wherein the polypeptide comprises or consists of a fragment of the amino acid sequence according to SEQ ID NO: 1

44. A method according to claim 43 wherein the fragment comprises or consists of at least 5 contiguous amino acid of SEQ ID NO: 1, for example at least 6, 7, 8, 9, 10, 11, 12, 13, 14, 15, 16, 17, 18, 19, 20, 21, 22, 23 or 24 contiguous amino acid of SEQ ID NO: 1.

45. A method according to Claim 43 wherein the fragment comprises or consists of amino acids 2 to 25 of SEQ ID NO: 1, for example amino acids 3 to 25, 4 to 25, 5 to 25, 6 to 25, 7 to 25, 8 to 25, 9 to 25, 10 to 25, 11 to 25, 12 to 25, 13 to 25, 14 to 25, to 25, 16 to 25, 17 to 25, 18 to 25, 19 to 25, 20 to 25 or 21 to 25 of SEQ ID NO: 1.

46. A method according to claim 43 wherein the fragment comprises or consists of amino acids 17 to 25 of SEQ ID NO. 1, for example amino acids 17 to 24, 17 to 23, 17 to 22, 17 to 21 or 17 to 20 of SEQ ID NO: 1.

47. A method according to claim 28 wherein the polypeptide comprises or consists of a variant of the amino acid sequence according to SEQ ID NO: 1.

48. A method according to claim 47 wherein the variant is a non-naturally occurring variant.

49. A method according to claim 47 wherein the variant has an amino acid sequence which has at least 45% identity with the amino acid sequence according to SEQ ID NO: 1 or a fragment thereof, for example at least 50%, 55%, 60%, 65%, 70%, 75%, 80%, 90%, 95%, 96%, 97%, 98% or at least 99% identity.

50. A method according to claim 28 wherein the polypeptide is a fusion protein.

51. A method according to claim 28 wherein the polypeptide comprises or consists of L-amino acids.

52. A method according to claim 28 wherein the polypeptide comprises one or more amino acids which are modified or derivatised.

53. A method according to claim 28 wherein the polypeptide, or fragment, variant, fusion or derivative thereof, is linear.

54. A method according to claim 28 wherein the polypeptide, or fragment, variant, fusion or derivative thereof, is cyclic.

55. A method according to claim 28 wherein the polypeptide, or fragment, variant, fusion or derivative thereof, is or comprises a recombinant polypeptide.


SEQUENCE LISTING (8 sequences)

SEQ ID NO: 1 - protein, 25 aa, Homo sapiens
LLGDFFRKSKEKIGKEFKRIVQRIK

SEQ ID NO: 2 - protein, 37 aa, Homo sapiens
LLGDFFRKSKEKIGKEFKRIVQRIKDFLRNLVPRTES

SEQ ID NO: 3 - DNA, 24 bp, Artificial Sequence
  Synthetic oligonucleotide hCAP18 qRT-PCR primer 1
gtcaccagaggattgtgacttcaa

SEQ ID NO: 4 - DNA, 21 bp, Artificial Sequence
  Synthetic oligonucleotide hCAP18 qRT-PCR primer 2
ttgagggtcactgtccccata

SEQ ID NO: 5 - DNA, 21 bp, Artificial Sequence
  Synthetic oligonucleotide hCAP18 fluorigenic probe
ccgcttcaccagcccgtcctt

SEQ ID NO: 6 - DNA, 22 bp, Artificial Sequence
  Synthetic oligonucleotide
tctgctggctacactgttctgc

SEQ ID NO: 7 - DNA, 19 bp, Artificial Sequence
  Synthetic oligonucleotide
gaccccgaggacaaaggtg

SEQ ID NO: 8 - DNA, 23 bp, Artificial Sequence
  Synthetic oligonucleotide
cccaagcaccaccaatgggagga